(19) United States
(12) Patent Application Publication
(10) Pub. No.: US 2025/0101344 A1
(43) Pub. Date: Mar. 27, 2025

(54) PHOSPHONATE-FREE CHELATING AGENTS AND METHODS FOR FABRIC CARE

(71) Applicant: The Procter & Gamble Company, Cincinnati, OH (US)

(72) Inventors: Sherri Lynn RANDALL, Hamilton, OH (US); Linsey Sarah FULLER, Newcastle Upon Tyne (GB); Renae Dianna FOSSUM, Middletown, OH (US); Mu WANG, Cincinnati, OH (US)

(21) Appl. No.: 18/890,838

(22) Filed: Sep. 20, 2024

Related U.S. Application Data
(60) Provisional application No. 63/584,666, filed on Sep. 22, 2023.

(51) Int. Cl. C11D 3/20; C07C 69/88; C11D 1/14; C11D 1/22; C11D 1/29; C11D 1/75; C11D 1/831; C11D 3/00; C11D 3/33; C11D 3/37; C11D 3/386

(52) U.S. Cl. CPC C11D 3/2093; C07C 69/88; C11D 1/831; C11D 3/0068; C11D 3/0084; C11D 3/2034; C11D 3/33; C11D 3/3788; C11D 3/38636; C11D 1/146; C11D 1/22; C11D 1/29; C11D 1/75

(57) ABSTRACT

Chelating agents may be incorporated into a variety of fabric care compositions and delivery formats. The fabric care compositions may include at least one component, according to Formula 7:

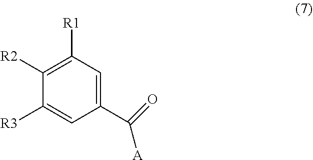

in which: R₁₋₃ is an OH or an H and at least 2 adjacent R's are OH; and A is a OR₄ or N(R₄)₂ or N(R₄)(H) or NH₂, where each R₄ is independently a linear or branched C1-18 carbon chain.

### FIELD

The present disclosure relates generally to chelating agents and methods for using chelating agents and more specifically to phosphonate-free chelating agents and methods.

### BACKGROUND

The discussion of shortcomings and needs existing in the field prior to the present disclosure is in no way an admission that such shortcomings and needs were recognized by those skilled in the art prior to the present disclosure.

Phosphonate chelants may be employed in various fabric care compositions to improve stain removal. Phosphonate chelants may improve stain removal via heavy metal chelation; in this regard, useful phosphonate chelants tend to have a higher binding efficiency with iron (III) than with calcium. One such phosphonate chelant is 1-Hydroxyethylidene-1,1-diphosphonic acid (HEDP), also known as etidronic acid or etidronate, as shown in Formula 1.

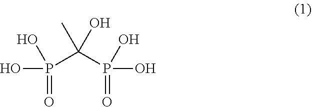

Another phosphonate chelant is diethylene triamine pentamethylene phosphonic acid (DTPMP) as shown in Formula 2.

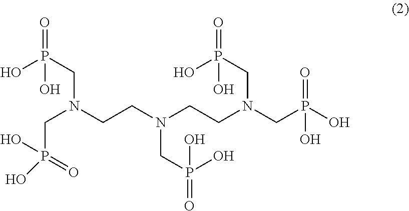 [SMILES: O=P(O)(O)CN(CCN(CP(=O)(O)O)CP(=O)(O)O)CCN(CP(=O)(O)O)CP(=O)(O)O]

Some phosphonate chelants are not readily biodegradable. In addition, alternative chelants to phosphonates are desirable as it allows for more options for formulation flexibility, for example. Therefore, a need exists to replace phosphonate chelants in various fabric care compositions, for example, with more biodegradable materials that preferably do not contain phosphorous.

Existing biodegradable replacements for phosphonate chelants include amino acid derivative complexing agents, as well as stereoisomers, and mixtures thereof. For example, methylglycinediacetic acid and salts thereof (MGDA), such as shown in Formula 3, has been used to replace phosphonate chelants.

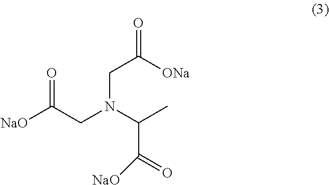

Similarly, L-glutamic acid, N,N-diacetic acid and salts thereof (GLDA) and salts thereof as shown in Formula 4, has been used to replace phosphonate chelants.

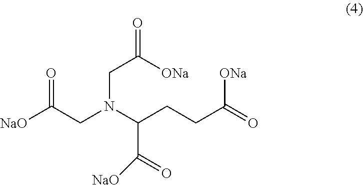

Unfortunately, neither MGDA nor GLDA match HEDP on stain removal performance. Therefore, a need still exists to replace phosphonate chelants in various fabric care compositions with materials that preferably do not contain phosphorous and are preferably biodegradable.

### SUMMARY

Various embodiments solve the above-mentioned problems and provide chelating agents that may be incorporated into a variety of fabric care compositions and delivery formats, and which provide similar or improved results compared to traditional chelating agents.

Various embodiments relate to detergent compositions comprising at least one component, according to Formula 7:

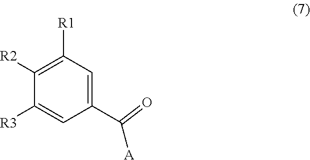

in which:
R₁₋₃ is an OH or an H and at least 2 adjacent R's are OH; and
A is an OR₄ or N(R₄)₂ or N(R₄)(H) or NH₂, where each R₄ is independently a linear or branched C1-18 carbon chain.
   An alkyl gallate suitable for use herein can include, for example, ethyl gallate, methyl gallate, propyl gallate, or a combination thereof.

These and other features, aspects, and advantages of various embodiments will become better understood with reference to the following description, figures, and claims.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such may vary. It is also to be understood that the terminology used herein is for purposes of describing particular embodiments only and is not intended to be limiting. It is also possible in the present disclosure that steps may be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

Unless otherwise specified, all percentages indicating the amount of a component in a composition represent a percent by weight of the component based on the total weight of the composition. The term “mol percent” or “mole percent” generally refers to the percentage that the moles of a particular component are of the total moles that are in a mixture. The sum of the mole fractions for each component in a solution is equal to 1.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent.

“Standard temperature and pressure” generally refers to 25° C. and 1 atmosphere. Standard temperature and pressure may also be referred to as “ambient conditions.” Unless indicated otherwise, parts are by weight, temperature is in ° C., and pressure is at or near atmospheric. The terms “elevated temperatures” or “high-temperatures” generally refer to temperatures of at least 100° C.

“Average size” refers to the particle size. The particle size of a spherical object may be unambiguously and quantitatively defined by its diameter. However, a typical material object is likely to be irregular in shape and non-spherical. There are several ways of extending the above quantitative definition to apply to non-spherical particles. Existing definitions are based on replacing a given particle with an imaginary sphere that has one of the properties identical with the particle. Volume-based particle size equals the diameter of the sphere that has the same volume as a given particle. Area-based particle size equals the diameter of the sphere that has the same surface area as a given particle. Weight-based particle size equals the diameter of the sphere that has the same weight as a given particle. Hydrodynamic or aerodynamic particle size equals the diameter of the sphere that has the same drag coefficient as a given particle.

“Mixing” refers to a unit operation in industrial process engineering that involves manipulation of a heterogeneous physical system with the intent to make it more homogeneous. Mixing is performed to allow heat and/or mass transfer to occur between one or more streams, components, or phases.

“Disposed on” refers to a positional state indicating that one object or material is arranged in a position adjacent to the position of another object or material. The term does not require or exclude the presence of intervening objects, materials, or layers.

“Compositions” include fabric care compositions for handwash, machine wash and/or other purposes and include fabric care additive compositions and compositions suitable for use in the soaking and/or pretreatment of fabrics. They may take the form of, for example, laundry detergents, fabric pre-treatments, fabric conditioners and/or other wash, rinse, dryer added products, and sprays. Compositions in the liquid form may be in an aqueous carrier. In other aspects, the fabric care compositions are in the form of a granular detergent or dryer added fabric softener sheet. The term includes, unless otherwise indicated, granular or powder-form all-purpose or “heavy-duty” washing agents, especially cleaning detergents; liquid, gel or paste-form all-purpose washing agents, especially the so-called heavy-duty liquid (HDL) types; liquid fine-fabric detergents; cleaning auxiliaries such as bleach additives and “stain-stick” or pre-treat types, substrate-laden products, dry and wetted wipes and pads, nonwoven substrates, and sponges; and sprays and mists. Various dosage formats may be used.

“Liquid” includes free-flowing liquids, as well as pastes, gels, foams and mousses. Non-limiting examples of liquids include light duty and heavy duty liquid detergent compositions, fabric enhancers, detergent gels commonly used for laundry, bleach and laundry additives. Gases, e.g., suspended bubbles, or solids, e.g. particles, may be included within the liquids.

“Solid” as used herein includes, but is not limited to, powders, agglomerates, and mixtures thereof. Non-limiting examples of solids include: granules, micro-capsules, beads, noodles, and pearlised balls. Solid compositions may provide a technical benefit including, but not limited to, through-the-wash benefits, pre-treatment benefits, and/or aesthetic effects.

It has unexpectedly been discovered that alkyl gallates, such as ethyl gallate, are able to match or to exceed HEDP performance in stain removal for various fabric care applications, like laundry detergents, and single unit dose fibrous applications. Various embodiments, therefore, meet the desire to replace phosphonate chelants in fabric care compositions with ones that do not contain phosphorous, preferably ones that are biodegradable.

Various embodiments relate to a chelating composition comprising at least one component, an alkyl gallate. Alkyl gallate is shown in Formula 7.

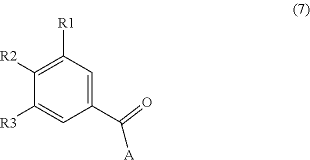

in which:
R₁₋₃ is an OH or an H and at least 2 adjacent R's are OH; and
A is a OR₄ or N(R₄)₂ or N(R₄)(H) or NH₂, where each R₄ is independently a linear or branched C1-18 carbon chain.

Chelation is a type of bonding of ions and molecules to metal ions. It involves the formation or presence of two or more separate coordinate bonds between a ligand and a metal atom. Without being bound by theory, the stain removal effectiveness of a composition comprising one or more chelating agents may depend at least in part on the ability of the chelating agent(s) to bind to iron, which may be present in some stains, such as fruit or beverage stains. Several factors may impact a chelating agent's ability to bind to iron. These factors may include the pKa of the chelating agent compared to the wash pH, and the affinity of the chelating agent to bind with other metals, materials, or compositions in the wash.

Various embodiments may provide or employ a composition that comprises one or more chelating agents having a pKa that is near to or less than the wash pH. For example, according to various embodiments, the chelating agent(s) may have a pKa of about 9.5 or less, about 9.0 or less, about 8, or less than about 8, or less than about 7.5, or less than about 7, or in a range of from about 7.0 to about 9.5. Various embodiments may also employ a wash liquor pH of about 10 or less, about 9.5 or less, less than about 9, preferably from about 6.5 to about 8.5, most preferably from about 7.0 to about 8.5. They may also include the step of maintaining the pH of the wash liquor at about 10 or less, about 9.5 or less, less than about 9, preferably from about 6.5 to about 8.5, most preferably from about 7.0 to about 8.5.

Various embodiments may provide or employ a composition that comprises one or more chelating agents exhibiting specific binding efficiencies with specific materials. The binding efficiencies may be expressed as a binding constant. A binding constant (also called formation constant or stability constant) is an equilibrium constant for the formation of a complex in solution. It is a measure of the strength of the interaction between the reagents that come together to form the complex. As an example, various stains may comprise catechins as shown in Formula 13.

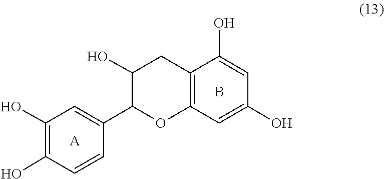

Such catechins may have a binding efficiency of about 3.5 with calcium and of about 19.2 with iron, as shown in Table 1, and are in competition with chelating agents to bind to these materials. The chelating agent(s) according to various embodiments may, therefore, be selected to ensure a lower binding efficiency with calcium and a higher binding efficiency with iron at the wash pH.

Various embodiments may provide or employ a composition that comprises one or more chelating agents having a calcium binding efficiency of about 2.0 to about 7.0, or about 2.2 to about 6.8, or about 2.5 to about 3.5, or about 2.7 to about 3.1, or less than about 6.8. A calcium binding efficiency of less than about 6.8, indicating that the chelating agent is less likely to bind to calcium than HEDP, as shown in Table 1.

Various embodiments may provide or employ a composition that comprises one or more chelating agents having an iron binding efficiency about 10 to about 20, or about 10.5 to about 14.1, or about 11 to about 12.9, greater than about 10, as shown in Table 1.

TABLE 1

| | | | Methyl | Ethyl |
|---|---|---|---|---|
| | Catechin | HEDP | Gallate | Gallate |
| pKa | 8.7 (A), | 1.7, 2.5, | 8.7, 11.4, | 8.7, 11.4, |
| | 9.5 (B), | 7.3, 10.3 | >13 | >13 |
| | 11.2 (B), | | | |
| | 13.4 (A) | | | |
| Ca²⁺ | 3.5 | 6.8 | 6.7 | 6.7 |
| logK(β₁₁₀) | | | | |
| Fe (III) | 19.2 | 14.1 | 14.7 | 14.7 |
| logK(β₁₁₀) | | | | |

In Table 1, the reaction between metals and ligands is described in terms of log K(β₁₁₀). In this context, K, is the equilibrium constant for the reaction between ligands and metals. The higher K, the more metal is bound. This is illustrated in the following example.

$$L {H}_{6} + {Fe}^{+ 3} {{K}_{\to }}_{←} L Fe + 6 {H}^{+}$$

$$K = \frac{{[ L Fe ] [ {H}^{+} ]}^{6}}{[ L {H}_{6} ] [ {Fe}^{+ 3} ]}$$

β, represents the binding efficiency and receives some subscripts (βₘₗₕ) to indicate how many metals (m), ligands (l), and protons (h) respectively are involved. Thus, β₁₁₀ indicates that one metal, one ligand, and no protons are involved.

### Detergent Composition

Various embodiments herein relate to a detergent composition. A detergent composition may be in the form of a liquid, gel, powder, bead, granule, fiber, or any combination thereof. Detergents may be dosed by hand, from a dispenser or from an automatic doing machine.

Various embodiments relate to a detergent composition comprising at least one component according to Formula 7. The at least one component may be present in an amount of from greater than about 0 to about 10% by weight, or about 0.5 to about 9.5% by weight, or about 0.5 to about 9.5% by weight, or about 1.0 to about 9.0% by weight, or about 1.5 to about 8.5% by weight, or about 2.0 to about 8.0% by weight, or about 2.5 to about 7.5% by weight, or about 3.0 to about 7.0% by weight, or about 3.5 to about 6.5% by weight, or about 4.0 to about 6.0% by weight, or about 4.5 to about 5.5% by weight, or about 4.0 to about 5.0%, or about 0.5% to about 2.0%, by weight of the detergent composition.

It is to be appreciated that the detergent composition may comprise a variety of other components in addition to the at least one component according to Formula 7. For example, the detergent composition may include a surfactant. The detergent composition may be a laundry detergent composition. A “laundry detergent composition” may include any composition intended for the cleaning of fabric in a washing machine or in a hand wash context. The laundry detergent compositions may be used in high efficiency and standard washing machines, in addition to hand washing in a tub or basin for example. It is to be appreciated that the detergent composition may encompass formulations that would be considered to be heavy duty liquid (HDL) detergent compositions or stain pre-treatment compositions, as those terms are used in the art.

The formulation may contain a preservative or a mixture of preservatives, selected from benzoic acid and salts thereof, alkyl esters of p-hydroxybenzoic acid and salts thereof, preferably benzoic acid and salts thereof, sodium benzoate, methylisothiazolinone, benzisothiazolinone, and 5-Chloro-2-Methyl-4-isothiazolinone. The preservative is present at 0.01 to 3 wt %, preferably 0.1 to 3 wt %, more preferably 0.3 wt % to 1.5 w %. Weights are calculated for the protonated form.

### Surfactant

The detergent composition may comprise from about 5% to about 75%, or about 5% to about 70%, or about 5% to about 65%, or about 5% to about 55%, or about 5% to about 50%, or about 10% to about 45%, or about 10% to about 40%, by weight of the detergent composition of a surfactant. The surfactant may be anionic, nonionic, amphoteric, zwitterionic, cationic, or a combination thereof.

Anionic surfactants may include, for example, alkylbenzene sulfonate, methyl ester sulfonate, alkyl ether carboxylate, alkyl sulfate, alkylalkoxylated sulfate, sodium lauryl sulfate, branched 2-alkyl primary alkyl alcohol sulfate, alkyl sulphate, or a combination thereof. The alkyl benzene sulfonate may comprise a linear alkylbenzene sulphonate. Linear alkylbenzene sulfonate, may have a small amount of branched alkylbenzene sulfonate as a byproduct of the manufacturing process, but this will generally be less than about 5%. The linear alkylbenzene sulphonate may be present, for example, at a level of 0.5% to about 30%, by weight of the detergent composition. The linear alkyl benzene sulphonate may be selected from, for example, alkyl benzene sulfonic acids, alkali metal or amine salts of C₁₀ to C₁₆ alkyl benzene sulfonic acids. In alkyl benzene sulfonic acids or alkali metal or amine salts of C₁₀ to C₁₆ alkyl benzene sulfonic acids, the linear alkyl benzene sulphonate surfactant can comprise greater than 50% C₁₂, greater than 60%, greater than 70% C₁₂, more preferably greater than 75%. The linear alkylbenzene sulphonate may comprise a C₁₀-C₁₆ alkyl benzene sulfonate, a C₁₁-C₁₄ alkyl benzene sulphonate, or a mixture thereof. The alkylbenzene sulphonate may be an amine neutralized alkylbenzene sulphonate, an alkali metal neutralized alkylbenzene sulphonate, or a mixture thereof. The amine comprises, for example, monoethanolamine, triethanolamine, monoisopropanolamine, or a mixture thereof. The alkali or alkali earth metal comprises, for example, sodium, potassium, magnesium, or a mixture thereof.

Another acceptable anionic surfactant comprises an alkyl sulphate anionic surfactant. The alkyl sulphate anionic surfactant may include, for example, alkyl sulphate, an alkoxylated alkyl sulphate, or a mixture thereof. The alkyl sulphate anionic surfactant may be a primary or a secondary alkyl sulphate anionic surfactant, or a mixture thereof, for example sodium lauryl sulfate. The alkoxylated alkyl sulphate may comprise an ethoxylated alkyl sulphate, propoxylated alkyl sulphate, a mixed ethoxylated/propoxylated alkyl sulphate, or a mixture thereof. An ethoxylated alkyl sulphate may have an average degree of ethoxylation of between 0.1 to 5, or between 0.5 and 3. The ethoxylated alkyl sulphate may have an average alkyl chain length of between 8 and 18, more preferably between 10 and 16, most preferably between 12 and 15. The alkyl portion of the ethoxylated alkyl sulphate may include, on average, from 13.7 to about 16 or from 13.9 to 14.6 carbons atoms. At least about 50% or at least about 60% of the AES molecule may include having an alkyl portion having 14 or more carbon atoms, from 14 to 18, or from 14 to 17, or from 14 to 16, or from 14 to 15 carbon atoms.

The alkyl chain of the alkyl sulphate anionic surfactant may be linear, branched or a mixture thereof. A branched alkyl sulphate anionic surfactant may be a branched primary alkyl sulphate, a branched secondary alkyl sulphate, or a mixture thereof, preferably a branched primary alkyl sulphate, wherein the branching preferably is in the 2-position, or alternatively might be present further down the alkyl chain or could be multi-branched with branches spread over the alkyl chain. The weight average degree of branching of alkyl sulphate anionic surfactant may be from 0% to 100% preferably from 0% to 95%, more preferably from 0% to 60%, most preferably from 0% to 20%. Alternatively, the weight average degree of branching of alkyl sulphate anionic surfactant may be from 70% to 100%, preferably from 80% to 90%. Preferably, the alkyl chain is selected from naturally derived material, synthetically derived material, or a mixture thereof. Preferably, the synthetically derived material comprises oxo-synthesized material, Ziegler-synthesized material, Guerbet-synthesized material, Fischer-Tropsch-synthesized material, iso-alkyl synthesized material, or mixtures thereof, preferably oxo-synthesized material.

Branched 2-alkyl primary alkyl alcohol sulfates and 2-alkyl primary alkyl alcohol ethoxy sulfates having specific alkyl chain length distributions, may provide increased stain removal (particularly in cold water). 2-alkyl branched alcohols (and the 2-alkyl branched alkyl sulfates and 2-alkyl branched alkyl ethoxy sulfates and other surfactants derived from them) are positional isomers, where the location of the hydroxymethyl group (consisting of a methylene bridge (—CH₂— unit) connected to a hydroxy (—OH) group) on the carbon chain varies. Thus, a 2-alkyl branched alkyl alcohol is generally composed of a mixture of positional isomers. Furthermore, it is well known that fatty alcohols, such as 2-alkyl branched alcohols, and surfactants are characterized by chain length distributions. In other words, fatty alcohols and surfactants are generally made up of a blend of molecules having different alkyl chain lengths (though it is possible to obtain single chain-length cuts). Notably, the 2-alkyl primary alcohols described herein, which may have specific alkyl chain length distributions and/or specific fractions of certain positional isomers, cannot be obtained by simply blending commercially available materials. Specifically, the distribution of from about 50% to about 100% by weight surfactants having m+n=11 is not achievable by blending commercially available materials.

The detergent composition may comprise a mixture of surfactant isomers of Formula 14 and surfactants of Formula 15:

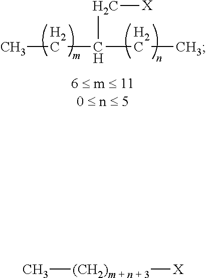

wherein from about 50% to about 100% by weight of the first surfactant are isomers having m+n=11; wherein from about 25% to about 50% of the mixture of surfactant isomers of Formula 14 have n=0; wherein from about 0.001% to about 25% by weight of the first surfactant are surfactants of Formula 15; and wherein X is a hydrophilic moiety.

X may be, for example, neutralized with sodium hydroxide, potassium hydroxide, magnesium hydroxide, lithium hydroxide, calcium hydroxide, ammonium hydroxide, monoethanolamine, diethanolamine, triethanolamine, monoisopropanolamine, diamine, polyamine, primary amine, secondary amine, tertiary amine, amine containing surfactant, or a combination thereof.

X may be selected from sulfates, alkoxylated alkyl sulfates, sulfonates, amine oxides, polyalkoxylates, polyhydroxy moieties, phosphate esters, glycerol sulfonates, polygluconates, polyphosphate esters, phosphonates, sulfosuccinates, sulfosuccaminates, polyalkoxylated carboxylates, glucamides, taurinates, sarcosinates, glycinates, isethionates, dialkanolamides, monoalkanolamides, monoalkanolamide sulfates, diglycolamides, diglycolamide sulfates, glycerol esters, glycerol ester sulfates, glycerol ethers, glycerol ether sulfates, polyglycerol ethers, polyglycerol ether sorbitan sulfates, esters, polyalkoxylated sorbitan esters, ammonioalkanesulfonates, amidopropyl betaines, alkylated quats, alkyated/polyhydroxyalkylated quats, alkylated/polyhydroxylated oxypropyl quats, imidazolines, 2-yl-succinates, sulfonated alkyl esters, sulfonated fatty acids, and mixtures thereof.

The alkyl ether carboxylate may be linear or branched. It may have an average carbon chain length of about 10 to about 26, about 10 to about 20, or about 16 to about 18. The alkyl ether carboxylate may have an average level of ethoxylation of about 2 to about 20, about 7 to about 13, about 8 to about 12, or about 9.5 to about 10.5. The acid form or salt form may be used. The alkyl chain may contain one cis or trans double bond. Commercial alkyl ether carboxylates are available, for example, from Kao (Akypo®), Huntsman (Empicol®), and Clariant (Emulsogen®).

The anionic surfactant may also be a biosurfactant. Anionic biosurfactants may include, for example, rhamnolipids. The rhamnolipid may have a single rhamnose sugar ring or two sugar rings.

The detergent composition may also comprise a non-ionic surfactant. A nonionic surfactant may comprise an alcohol alkoxylate, an oxo-synthesized alcohol alkoxylate, a Guerbet alcohol alkoxylate, an alkyl phenol alcohol alkoxylate, an alkylpolyglucoside, or a mixture thereof. Preferably, a non-ionic surfactant may include, for example, alkoxylated alcohol nonionic surfactants, alkyl polyglucoside nonionic surfactants, and mixtures thereof. Preferably, the alkoxylated alcohol non-ionic surfactant is a linear or branched, primary or secondary alkyl alkoxylated non-ionic surfactant, preferably an alkyl alkoxylated non-ionic surfactant, preferably an alkyl ethoxylated non-ionic surfactant, preferably comprising on average from about 9 to about 15, preferably from about 10 to about 16, more preferably from about 12 to about 15, carbon atoms in its alkyl chain; and on average from about 5 to about 12, preferably from about 6 to about 10, most preferably from about 7 to about 8 or from about 9 to about 10, units of ethylene oxide per mole of alcohol. For example, a non-ionic surfactant can comprise an ethoxylated non-ionic surfactant wherein the ethoxylated nonionic surfactant with an average carbon chain length of about 10 to about 16 comprises an ethoxylated nonionic surfactant with an average carbon chain length of about 12 to about 14 and an average level of ethoxylation of about 9 and a second ethoxylated nonionic surfactant with an average carbon chain length of about 14 to about 15 and an average ethoxylation of about 7.

The nonionic surfactant may have the formula R(OC₂H₄)ₙOH, wherein R is selected from the group consisting of aliphatic hydrocarbon radicals containing from about 8 to about 16 carbon atoms and can be linear or branched and the average value of n is from about 5 to about 15. For example, the additional nonionic surfactant may be selected from ethoxylated alcohols having an average of about 12-14 carbon atoms in the alcohol (alkyl) portion and an average degree of ethoxylation of about 7-9 moles of ethylene oxide per mole of alcohol.

Additional non limiting examples include ethoxylated alkyl phenols of the formula R(OC₂H₄)ₙOH, wherein R comprises an alkyl phenyl radicals in which the alkyl groups contain from about 8 to about 12 carbon atoms, and the average value of n is from about 5 to about 15, C₁₂-C₁₈ alkyl ethoxylates, such as, NEODOL® nonionic surfactants from Shell; C₁₄-C₂₂ mid-chain branched alcohols; C₁₄-C₂₂ mid-chain branched alkyl ethoxylates, BAEₓ, wherein x is from 1 to 30. The nonionic ethoxylated alcohol surfactant herein may further comprise residual alkoxylation catalyst, which may be considered residue from the reaction or an impurity. It may further comprise various impurities or by-products of the alkoxylation reaction. The impurities may vary depending on the catalyst used and the conditions of the reaction. Impurities include alkyl ethers, e.g., dialkyl ethers, such as, didodecyl ether, glycols, e.g., diethylene glycol, triethylene glycol, pentaethylene glycol, other polyethylene glycols.

The nonionic ethoxylated alcohol may be a narrow range ethoxylated alcohol. A narrow range ethoxylated alcohol may have the following general formula (I):

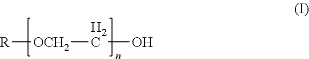

where R is selected from a saturated or unsaturated, linear or branched, C₁₀-C₁₆ alkyl group and where greater than 90% of n is 0≤n≤15. In addition, the average value of n can be between about 4 to about 14, preferably about 6 to about 10, where less than about 10% by weight of the alcohol ethoxylate are ethoxylates having n<7 and between 10% and about 20% by weight of the alcohol ethoxylate are ethoxylates having n=8.

The composition may comprise an average value of n of about 10. The composition may have the following ranges for each of the following n: n=0 of up to 5%, each of n=1, 2, 3, 4, 5 of up to 2%, n=6 of up to 4%, n=7 of up to 10%, n=8 of between 12% and 20%, n=9 of between 15% and 25%, n=10 of between 15% to 30%, n=11 of between 10% and 20%, n=12 of up to 10%, and n>12 at up to 10%. The composition may have n=9 to 10 of between 30% and 70%. The composition may have greater than 50% of its composition made up of n=8 to 11.

R can be selected from a saturated or unsaturated, linear or branched, C10-C16 alkyl group, where the average value of n is between about 6 and about 10. R can also be selected from a saturated or unsaturated, linear or branched, C₈-C₁₆ alkyl group, where greater than 90% of n is 0≤n≤15, and where the average value of n between about 5 to about 10, where less than about 20% by weight of the alcohol ethoxylate are ethoxylates having n<8. R can also be selected from a saturated or unsaturated, linear or branched, C₁₀-C₁₆ alkyl group, where greater than 90% of n is 0≤n≤15, and where the average value of n between about 6 to about 10, where less than about 10% by weight of the alcohol ethoxylate are ethoxylates having n<7 and between 10% and about 20% by weight of the alcohol ethoxylate are ethoxylates having n=8.

The alcohol ethoxylates described herein are typically not single compounds as suggested by their general formula (I), but rather, they comprise a mixture of several homologs having varied polyalkylene oxide chain length and molecular weight. Among the homologs, those with the number of total alkylene oxide units per mole of alcohol closer to the most prevalent alkylene oxide adduct are desirable; homologs whose number of total alkylene oxide units is much lower or much higher than the most prevalent alkylene oxide adduct are less desirable. In other words, a “narrow range” or “peaked” alkoxylated alcohol composition is desirable. A “narrow range” or “peaked” alkoxylated alcohol composition refers to an alkoxylated alcohol composition having a narrow distribution of alkylene oxide addition moles.

A “narrow range” or “peaked” alkoxylated alcohol composition may be desirable for a selected application. Homologs in the selected target distribution range may have the proper lipophilic-hydrophilic balance for a selected application. For example, in the case of an ethoxylated alcohol product comprising an average ratio of 5 ethylene oxide (EO) units per molecule, homologs having a desired lipophilic-hydrophilic balance may range from 2EO to 9EO. Homologs with shorter EO chain length (<2EO) or longer EO chain length (>9EO) may not be desirable for the applications for which a=5 EO/alcohol ratio surfactant is ordinarily selected since such longer and shorter homologs are either too lipophilic or too hydrophilic for the applications utilizing this product. Therefore, it is advantageous to develop an alkoxylated alcohol having a peaked distribution.

The narrow range alkoxylated alcohol compositions of the disclosure may have an average degree of ethoxylation ranging from about 0 to about 15, such as, for example, ranging from about 4 to about 14, from about 5-10, from about 8-11, and from about 6-9. The narrow range alkoxylated alcohol compositions of the disclosure may have an average degree of ethoxylation of 10. The narrow range alkoxylated alcohol compositions of the disclosure may have an average degree of ethoxylation of 9. The narrow range alkoxylated alcohol compositions of the disclosure may have an average degree of ethoxylation of 5.

The alkyl polyglucoside surfactant can be selected from C10-C16 alkyl polyglucoside surfactant. The alkyl polyglucoside surfactant can have a number average degree of polymerization of from 0.1 to 3.0, preferably from 1.0 to 2.0, more preferably from 1.2 to 1.6. The alkyl polyglucoside surfactant can comprise a blend of short chain alkyl polyglucoside surfactant having an alkyl chain comprising 10 carbon atoms or less, and mid to long chain alkyl polyglucoside surfactant having an alkyl chain comprising greater than 10 carbon atoms to 18 carbon atoms, preferably from 12 to 14 carbon atoms.

Short chain alkyl polyglucoside surfactants have a monomodal chain length distribution between C8-C10, mid to long chain alkyl polyglucoside surfactants have a monomodal chain length distribution between C10-C18, while mid chain alkyl polyglucoside surfactants have a monomodal chain length distribution between C12-C14. In contrast, C8 to C18 alkyl polyglucoside surfactants typically have a monomodal distribution of alkyl chains between C8 and C18, as with C8 to C16 and the like. As such, a combination of short chain alkyl polyglucoside surfactants with mid to long chain or mid chain alkyl polyglucoside surfactants have a broader distribution of chain lengths, or even a bimodal distribution, than non-blended C8 to C18 alkyl polyglucoside surfactants. Preferably, the weight ratio of short chain alkyl polyglucoside surfactant to long chain alkyl polyglucoside surfactant is from 1:1 to 10:1, preferably from 1.5:1 to 5:1, more preferably from 2:1 to 4:1. It has been found that a blend of such short chain alkyl polyglucoside surfactant and long chain alkyl polyglucoside surfactant results in faster dissolution of the detergent solution in water and improved initial sudsing, in combination with improved suds stability.

C10-C16 alkyl polyglucosides are commercially available from several suppliers (e.g., Simusol® surfactants from Seppic Corporation; and Glucopon® 600 CSUP, Glucopon® 650 EC, Glucopon® 600 CSUP/MB, and Glucopon® 650 EC/MB, from BASF Corporation). Glucopon® 215UP is a preferred short chain APG surfactant. Glucopon® 600CSUP is a preferred mid to long chain APG surfactant.

The detergent composition may also comprise an amphoteric surfactant and/or zwitterionic surfactant. Suitable amphoteric or zwitterionic surfactants include amine oxides, and/or betaines. Preferred amine oxides are alkyl dimethyl amine oxide or alkyl amidopropyl dimethyl amine oxide, more preferably alkyl dimethyl amine oxide and especially coco dimethyl amine oxide. Amine oxide may have a linear or mid-branched alkyl moiety. Typical linear amine oxides include water-soluble amine oxides containing one R₄ C₈ to C₁₈ alkyl moiety and 2 R₅ and R₆ moieties selected from the group consisting of C₁ to C₃ alkyl groups and C₁ to C₃ hydroxyalkyl groups. Preferably amine oxide is characterized by Formula 16

R₄—N(R₅)(R₆)O (16)

in which R₄ is a C₈ to C₁₈ alkyl and R₅ and R₆ are selected from the group consisting of methyl, ethyl, propyl, isopropyl, 2-hydroxethyl, 2-hydroxypropyl and 3-hydroxypropyl. The linear amine oxide surfactants in particular may include linear C₁₀-C₁₈ alkyl dimethyl amine oxides and linear C₈-C₁₂ alkoxy ethyl dihydroxy ethyl amine oxides.

Other suitable surfactants include betaines, such as alkyl betaines, alkylamidobetaine, amidazoliniumbetaine, sulfobetaine (sultaines) as well as phosphobetaines, or a combination thereof.

Suitable cationic surfactants include alkyl pyridinium compounds, alkyl quaternary ammonium compounds, alkyl quaternary phosphonium compounds, alkyl ternary sulphonium compounds, and mixtures thereof.

Preferred cationic surfactants are quaternary ammonium compounds having the general formula:

(R)(R₁)(R₂)(R₃)N⁺X⁻

wherein, R is a linear or branched, substituted or unsubstituted C₆₋₁₈ alkyl or alkenyl moiety, R₁ and R₂ are independently selected from methyl or ethyl moieties, R₃ is a methyl, hydroxyl, hydroxymethyl or a hydroxyethyl moiety, X is an anion which provides charge neutrality, preferred anions include: halides, preferably chloride; sulphate; and sulphonate.

The fabric care compositions of the present invention may contain up to about 30%, alternatively from about 0.01% to about 20%, more alternatively from about 0.1% to about 20%, by weight of the composition, of a cationic surfactant. For the purposes of the present invention, cationic surfactants include those which can deliver fabric care benefits, including but not limited to C₁₀-C₁₆ trimethyl ammonium chloride.

The quaternary ammonium compounds of the present disclosure may comprise one or members selected from the group consisting of:
(A) bis-(2-hydroxypropyl)-dimethylammonium methylsulfate fatty acid ester and isomers of bis-(2-hydroxypropyl)-dimethylammonium methylsulfate fatty acid ester and/or mixtures thereof; N,N-bis-(2-(acyl-oxy)-propyl)-N,N-dimethylammonium methylsulfate and/or N-(2-(acyl-oxy)-propyl)N-(2-(acyl-oxy) 1-methyl-ethyl) N,N-dimethylammonium methylsulfate and/or mixtures thereof, in which the acyl moiety is derived from c12-c22 fatty acids such as Palm, Tallow, Canola and/or other suitable fatty acids, which can be fractionated and/or hydrogenated, and/or mixtures thereof;
(B) 1,2-di(acyloxy)-3-trimethylammoniopropane chloride in which the acyl moiety is derived from c12-c22 fatty acids such as Palm, Tallow, Canola and/or other suitable fatty acids, which can be fractionated and/or hydrogenated, and/or mixtures thereof;
(C) N,N-bis(hydroxyethyl)-N,N-dimethyl ammonium chloride fatty acid esters; N,N-bis(acyl-oxy-ethyl)-N,N-dimethyl ammonium chloride in which the acyl moiety is derived from C12-C22 fatty acids such as Palm, Tallow, Canola and/or other suitable fatty acids, which can be fractionated and/or hydrogenated, and/or mixtures thereof, such as N,N-bis(tallowoyl-oxy-ethyl) N,N-dimethyl ammonium chloride;
(D) esterification products of Fatty Acids with Triethanolamine, quaternized with Dimethyl Sulphate; N,N-bis(acyl-oxy-ethyl)N-(2-hydroxyethyl)-N-methyl ammonium methylsulfate in which the acyl moiety is derived from C12-C22 fatty acids such as Palm, Tallow, Canola, Oleic, rapseed and/or other suitable fatty acids, which can be fractionated and/or hydrogenated, and/or mixtures thereof, such as N,N-bis(tallowoyl-oxy-ethyl)N-(2-hydroxyethyl)-N-methyl ammonium methylsulfate;
(E) dicanoladimethylammonium chloride; di(hard)tallowdimethylammonium chloride; dicanoladimethylammonium methylsulfate; 1-methyl-1-stearoylamidoethyl-2-stearoylimidazolinium methylsulfate; 1-tallowylamidoethyl-2-tallowylimidazoline; dipalmylmethyl hydroxyethylammoinum methylsulfate; and/or
(F) mixtures thereof.

Examples of suitable quaternary ammonium ester compound are commercially available from Evonik under the tradename REWOQUAT WE18 and/or REWOQUAT WE20, and from Stepan under the tradename STEPANTEX GA90, STEPANTEX VK90, and/or STEPANTEX VL90A.

It is understood that compositions that comprise a quaternary ammonium ester compound as a fabric conditioning active may further comprise non-quaternized derivatives of such compounds, as well as unreacted reactants (e.g., free fatty acids).

The quaternary ammonium compound can be the reaction product of triethanolamine and partially hydrogenated tallow fatty acids quaternized with dimethyl sulfate.

The composition may also comprise a softening active such as polyglycerol esters (PGEs), oily sugar derivatives, and wax emulsions and a mixture of the above.

It will be understood that combinations of softener actives disclosed above are suitable for use herein.

### Graft Copolymer

The compositions and methods of the present disclosure relate to a graft polymer. Broadly, the graft polymer may comprise and/or be obtainable by grafting (a) a polyalklyene oxide with (b) N-vinylpyrrolidone and (c) a vinyl ester. The graft polymer is described in more detail below.

Compositions according to the present disclosure may include from about 0.1% to about 50%, or to about 40%, or to about 25%, or from about 0.1% to about 15%, or from about 0.1% to about 10%, or from about 0.2% to about 5%, by weight of the composition, of the graft polymer. The graft polymer may be present in an aqueous treatment liquor, such as a wash liquor or a rinse liquor of an automatic washing machine, in an amount of about 5 ppm, or from about 10 ppm, or from about 25 ppm, or from about 50 ppm, to about 1500 ppm, or to about 1000 ppm, or to about 500 ppm, or to about 250 ppm.

The graft polymer may be comprise and/or be obtainable by grafting (a) a polyalkylene oxide which has a number average molecular weight of from about 1000 to about 20,000, or to about 15,000, or to about 12,000, or to about 10,000 Daltons and is based on ethylene oxide, propylene oxide, or butylene oxide, preferably based on ethylene oxide, with (b) N-vinylpyrrolidone, and further with (c) a vinyl ester derived from a saturated monocarboxylic acid containing from 1 to 6 carbon atoms and/or a methyl or ethyl ester of acrylic or methacrylic acid, preferably a vinyl ester that is vinyl acetate or a derivative thereof; where the weight ratio of (a):(b) is from about 1:0.1 to about 1:1; where the amount, by weight, of (a) is greater than the amount of (c); and where the order of the addition of monomers (b) and (c) in the graft polymerization is immaterial.

The graft polymer may comprise and/or be obtainable by grafting (a) an alkylene oxide which has a number average molecular weight of from about 1000 to 20,000, or to about 15,000, or to about 12,000, or to about 10,000 Daltons, the alkylene oxide being based on ethylene oxide, with (b) N-vinylpyrrolidone, and (c) vinyl acetate or a derivative thereof; wherein the weight ratio of (a):(b) is from about 1:0.1 to about 1:2, or to about 1:1; wherein the weight ratio of (b):(c) is from about 1:0.1 to about 1:5, or to about 1:4; wherein the weight ratio of (a):(c) is from about 1:0.1 to about 1:5, or to about 1:3; the order of the addition of monomers (b) and (c) in the graft polymerization being immaterial.

The graft polymer may be obtainable by grafting (a) an alkylene oxide which has a number average molecular weight of from about 1000 to 20,000, or to about 15,000, or to about 12,000, or to about 10,000 Daltons, the alkylene oxide being based on ethylene oxide, with (b) N-vinylpyrrolidone, and (c) vinyl acetate or a derivative thereof, the order of the addition of monomers (b) and (c) in the graft polymerization being immaterial, wherein the number of grafting sites is less than 1 per 50 ethylene oxide groups, wherein the composition is a fabric care composition.

The graft bases used may be the polyalkylene oxides specified above under (a). The polyalkylene oxides of component (a) may have a number average molecular weight of about 300, or from about 1000, or from about 2000, or from about 3,000, to about 20,000, or to about 15,000, or to about 12,000, or to about 10,000, or to about 8,000, or to about 6,000 Daltons (Da). Without wishing to be bound by theory, it is believed that if the molecular weight of component (a) (e.g., polyethylene glycol), is relatively low, there may be a performance decrease in dye transfer inhibition. Additionally or alternatively, when the molecular weight is too high, the polymer may not remain suspended in solution and/or may deposit on treated fabrics.

The polyalkylene oxides may be based on ethylene oxide, propylene oxide, butylene oxides, or mixtures thereof, preferably ethylene oxide. The polyalkylene oxides may be based on homopolymers of ethylene oxide or ethylene oxide copolymers having an ethylene oxide content of from about 40 to about 99 mole %. Suitable comonomers for such copolymers may include propylene oxide, n-butylene oxide, and/or isobutylene oxide. Suitable copolymers may include copolymers of ethylene oxide and propylene oxide, copolymers of ethylene oxide and butylene oxide, and/or copolymers of ethylene oxide, propylene oxide, and at least one butylene oxide. The copolymers may include an ethylene oxide content of from about 40 to about 99 mole %, a propylene oxide content of from about 1 to about 60 mole %, and a butylene oxide content of from about 1 to about 30 mole %. The graft base may be linear (straight-chain) or branched, for example a branched homopolymer and/or a branched copolymer.

Branched copolymers may be prepared by addition of ethylene oxide with or without propylene oxides and/or butylene oxides onto polyhydric low molecular weight alcohols, for example trimethylol propane, pentoses, or hexoses. The alkylene oxide unit may be randomly distributed in the polymer or be present therein as blocks.

The polyalkylene oxides of component (a) may be the corresponding polyalkylene glycols in free form, i.e, with OH end groups, or they may be capped at one or both end groups. Suitable end groups may be, for example, C1-C25-alkyl, phenyl, and C1-C14-alkylphenyl groups. The end group may be a C1-alkyl (e.g., methyl) group. Suitable materials for the graft base may include PEG 300, PEG 1000, PEG 2000, PEG 4000, PEG 6000, PEG 8000, and/or PEG 10,000 which are polyethylene glycols, and/or MPEG 2000, MPEG 4000, MPEG 6000, MPEG 8000 and MEG 10000 which are monomethoxypolyethylene glycols that are commercially available from BASF under the tradename Pluriol®.

The polyalkylene oxides may be grafted with N-vinylpyrrolidone as the monomer of component (b). Without wishing to be bound by theory, it is believed that the presence of the N-vinylpyrrolidone (“VP”) monomer in the graft polymers according to the present disclosure provides water-solubility and good film-forming properties compared to otherwise-similar polymers that do not contain the VP monomer. The vinyl pyrrolidone repeat unit has amphiphilic character with a polar amide group that can form a dipole, and a non-polar portion with the methylene groups in the backbone and the ring, making it hydrophobic. When the vinyl pyrrolidone content is too high, there may be negative interactions with other ingredients in the detergent such as brightener causing physical instability, and material cost is high with high vinyl pyrrolidone content.

The polyalkylene oxides may be grafted with a vinyl ester as the monomer of component (c). The vinyl ester may be derived from a saturated monocarboxylic acid, which may contain 1 to 6 carbon atoms, or from 1 to 3 carbon atoms, or from 1 to 2 carbon atoms, or 1 carbon atom. The vinyl ester may be derived from methyl acrylate, ethyl acrylate, methyl methacrylate, ethyl methacrylate, or mixtures thereof. Suitable vinyl esters may include vinyl formate, vinyl acetate, vinyl propionate, vinyl butyrate, vinyl valerate, vinyl iso-valerate, vinyl caproate, or mixtures thereof. Preferred monomers of component (c) include vinyl acetate, vinyl propionate, methyl acrylate, mixtures of vinyl acetate and methyl acrylate, or mixtures thereof, preferably vinyl acetate. The monomers of the graft polymer, e.g., components (a), (b), and/or (c), may be present in certain ratios, such as weight ratios and/or mole ratios.

For example, the weight ratio of (a):(b) may be from about 1:0.1 to about 1:1, or from about 1:0.2 to about 1:0.7. The weight ratio of (a):(b) may be from about 1:0.1 to about 1:2, or to about 1:1. When the VP ratio is too high, the polymer may form negative interactions with other detergent ingredients such as brightener, and/or may not work sufficiently on some hydrolyzed reactive dyes.

The weight ratio of (a):(c) may be greater than 1:1, or from about 1:0.1 to about 1:0.8, or from about 1:0.2 to about 1:0.6. The weight ratio of (a):(c) is from about 1:0.1 to about 1:5, or to about 1:3. The amount, by weight, of (a) may be greater than the amount of (c). Without wishing to be bound by theory, it is believed that relatively high levels of component (c) (e.g., vinyl acetate), particularly in relation to component (a), may result in decreased performance of dye transfer inhibition and/or relatively greater hydrophobicity, which can lead to formulation and/or stability challenges.

The weight ratio of (b):(c) may be from about 1:0.1 to about 1:5, or to about 1:4. Without wishing to be bound by theory, a ratio of VP-to-VAc that is too high may lead to treated fabric having a negative feel. Additionally, negative interactions with ingredients such as brighteners may occur.

The graft polymers of the present disclosure may be characterized by relatively low degree of branching (i.e., degree of grafting). In the graft polymers of the present disclosure, the average number of grafting sites may be less than or equal to 1, or less than or equal to 0.8, or less than or equal to 0.6, or less than or equal to 0.5, or less than or equal to 0.4, per 50 alkylene oxide groups, e.g., ethylene oxide groups. The graft polymers may comprise, on average, based on the reaction mixture obtained, at least 0.05, or at least 0.1, graft site per 50 alkylene oxide groups, e.g., ethylene oxide groups. The degree of branching may be determined, for example, by means of ¹³C NMR spectroscopy from the integrals of the signals of the graft sites and the —CH₂-groups of the polyakylene oxide. The number of grafting sites may be adjusted by manipulating the temperature and/or the feed rate of the monomers. For example, the polymerization may be carried out in such a way that an excess of component (a) and the formed graft polymer is constantly present in the reactor. For example, the quantitative molar ratio of component (a) and polymer to ungrafted monomer (and initiator, if any) is generally greater than or equal to about 10:1, or to about 15:1, or to about 20:1.

The graft polymers of the present disclosure may be characterized by a relatively narrow molar mass distribution. For example, the graft polymers may be characterized by a polydispersity M_{w}/Mₙ of less than or equal to about 3, or less than or equal to about 2.5, or less than or equal to about 2.3. The polydispersity of the graft polymers may be from about 1.5 to about 2.2. The polydispersity may be determined by gel permeation chromatography using narrow-distribution polymethyl methacrylates as the standard.

The graft polymers may be prepared by grafting the suitable polyalkylene oxides of component (a) with the monomers of component (b) in the presence of free radical initiators and/or by the action of high-energy radiation, which may include the action of high-energy electrons. This may be done, for example, by dissolving the polyalkylene oxide in at least one monomer of group (b), adding a polymerization initiator and polymerizing the mixture to completion. The graft polymerization may also be carried out semicontinuously by first introducing a portion, for example 10%, of the mixture of polyalkylene oxide to be polymerized, at least one monomer of group (b) and/or (c) and initiator, heating to polymerization temperature and, after the polymerization has started, adding the remainder of the mixture to be polymerized at a rate commensurate with the rate of polymerization. The graft polymers may also be obtained by introducing the polyalkylene oxides of group (a) into a reactor, heating to the polymerization temperature, and adding at least one monomer of group (b) and/or (c) and polymerization initiator, either all at once, a little at a time, or uninterruptedly, preferably uninterruptedly, and polymerizing.

In the preparation of the graft polymers, the order in which the monomers (b) and (c) are grafted onto component (a) may be immaterial and/or freely chooseable. For example, first N-vinylpyrrolidone may be grafted onto component (a), and then a monomer (c) or a mixture of monomers of group (c). It is also possible to first graft the monomers of group (c) and then N-vinylpyrrolidone onto the graft base (a). It may be that a monomer mixture of (b) and (c) are grafted onto graft base (a) in one step. The graft polymer may be prepared by providing graft base (a) and then first grafting N-vinylpyrrolidone and then vinyl acetate onto the graft base.

Any suitable polymerization initiator(s) may be used, which may include organic peroxides such as diacetyl peroxide, dibenzoyl peroxide, succinyl peroxide, di-tert-butyl peroxide, tert-butyl perbenzoate, tert-butyl perpivalate, tert-butyl permaleate, cumene hydroperoxide, diisopropyl peroxodicarbamate, bis(o-toluoyl) peroxide, didecanoyl peroxide, dioctanoyl peroxide, dilauroyl peroxide, tert-butyl perisobutyrate, tert-butyl peracetate, di-tert-amyl peroxide, tert-butyl peracetate, di-tert-amyl peroxide, tert-butyl hydroperoxide, mixtures thereof, redox initiators, and/or azo starters. The choice of initiator may be related to the choice of polymerization temperature.

The graft polymerization may take place at from about 50° C. to about 200° C., or from about 70° C. to about 140° C. The graft polymerization may typically be carried out under atmospheric pressure, but may also be carried out under reduced or superatmospheric pressure.

The graft polymerization may be carried out in a solvent. Suitable solvents may include: monohydric alcohols, such as ethanol, propanols, and/or butanols; polyhydric alcohols, such as ethylene glycol and/or propylene glycol; alkylene glycol ethers, such as ethylene glycol monomethyl and -ethyl ether and/or propylene glycol monomethyl and -ethyl ether; polyalkylene glycols, such as di- or tri-ethylene glycol and/or di- or tri-propylene glycol; polyalkylene glycol monoethers, such as poly(C2-C3-alkylene) glycol mono(C1-C16-alkyl) ethers having 3-20 alkylene glycol units; carboxylic esters, such as ethyl acetate and ethyl propionate; aliphatic ketones, such as acetone and/or cyclohexanone; cyclic ethers, such as tetrahydrofuran and/or dioxane; or mixtures thereof.

The graft polymerization may also be carried out in water as solvent. In such cases, the first step may be to introduce a solution which, depending on the amount of added monomers of component (b) is more or less soluble in water. To transfer water-insoluble products that can form during the polymerization into solution, it is possible, for example, to add organic solvents, for example monohydric alcohols having 1 to 3 carbon atoms, acetone, and/or dimethylformamide. In a graft polymerization process in water, it is also possible to transfer the water-insoluble graft polymers into a finely divided dispersion by adding customary emulsifiers or protective colloids, for example polyvinyl alcohol. The emulsifiers used may be ionic or nonionic surfactants whose HLB value is from about 3 to about 13. HLB value is determined according to the method described in the paper by W. C. Griffin in J. Soc. Cosmet. Chem. 5 (1954), 249.

The amount of surfactant used in the graft polymerization process may be from about 0.1 to about 5% by weight of the graft polymer. If water is used as the solvent, solutions or dispersions of graft polymers may be obtained. If solutions of graft polymers are prepared in an organic solvent or in mixtures of an organic solvent and water, the amount of organic solvent or solvent mixture used per 100 parts by weight of the graft polymer may be from about 5 to about 200, preferably from about 10 to about 100, parts by weight.

The graft polymers may have a K value of from about 5 to about 200, preferably from about 5 to about 50, determined according to H. Fikentscher in 2% strength by weight solution in dimethylformamide at 25 C.

After the graft polymerization, the graft polymer may optionally be subjected to a partial hydrolysis. The graft polymer may include up to 60 mole %, or up to 50 mole %, or up to 40 mole %, or up to 25 mole %, or up to 20 mole %, or up to 15 mole %, or up to 10 mole %, of the grafted-on monomers of component (c) are hydrolyzed. For instance, the hydrolysis of graft polymers prepared using vinyl acetate or vinyl propionate as component (c) gives graft polymers containing vinyl alcohol units. The hydrolysis may be carried out, for example, by adding a base, such as sodium hydroxide solution or potassium hydroxide solution, or alternatively by adding acids and if necessary heating the mixture. Without wishing to be bound by theory, it is believed that increasing the level of hydrolysis of component (c) increases the relative hydrophilicity of the graft polymer, which in turn is believed to result in better suspension of the captured dyes.

### Perfume

The detergent composition may also comprise a perfume. Perfume may be present, for example, at a level of about 0.05% to about 6.0%, or from about 0.1 to about 5.0%, or from about 0.1 to about 4%, or from about 0.1 to about 3%, or from about 0.1 to about 2.0%.

### Water

The detergent composition also includes water. Water may be present, for example, at a level of about 5% to about 95%, by weight of the composition.

pH

The detergent composition may have a pH of about 5.0 to about 12, preferably 6.0-9.0.0, more preferably from 6.5 to 8.5, wherein the pH of the detergent composition is measured as a 10% dilution in demineralized water at 20° C.

### Adjunct Ingredients

Compositions according to various embodiments may comprise one or more adjunct ingredient(s). The non-limiting list of adjuncts provided hereinafter are suitable for use in the instant compositions and may be desirably incorporated in certain embodiments, for example to assist or enhance performance, for treatment of the substrate to be cleaned, or to modify the aesthetics of the composition as is the case with colorants, dyes or the like. Suitable adjunct materials include, but are not limited to, polymers, for example cationic polymers, surfactants, builders, dye transfer inhibiting agents, dispersants, enzymes, and enzyme stabilizers, catalytic materials, bleach activators, polymeric dispersing agents, clay soil removal/anti-redeposition agents, brighteners, suds suppressors, dyes, additional perfume and perfume delivery systems, structure elasticizing agents, fabric softeners, carriers, hydrotropes, processing aids and/or pigments.

The detergent composition may comprise one or more adjunct ingredients. Adjunct ingredients may include, for example, color care agents; organic solvents; aesthetic dyes; hueing dyes; leuco dyes; opacifiers such as those commercially available under the Acusol tradename, brighteners including FWA49, FWA15, and FWA36; dye transfer inhibitors including PVNO, PVP and PVPVI dye transfer inhibitors; builders including citric acid- and fatty acids; other chelants (i.e., chelants other than those according to Formula 7); enzymes; perfume capsules; preservatives; antioxidants including sulfite salts such as potassium sulphite or potassium bisulphite salts and those commercially available under the Ralox brand name; antibacterial and anti-viral agents including 4,4′-dichloro 2-hydroxydiphenyl ether such as Tinosan HP100 available from the BASF company; anti-mite actives such as benzyl benzoate; structuring agents including hydrogenated castor oil; silicone based anti-foam materials; electrolytes including inorganic electrolytes such as sodium chloride, potassium chloride, magnesium chloride, and calcium chloride, and related sodium, potassium, magnesium and calcium sulphate salts, as well as organic electrolytes such as sodium, potassium, magnesium and calcium salts of carbonate, bicarbonate, carboxylates such as formate, citrate and acetate; pH trimming agents including sodium hydroxide, hydrogen chloride, and alkanolamines including monoethanolamine, diethanolamine, triethanolamine, and monoisopropanolamine; a probiotic; a hygiene agent such as zinc ricinoleate, thymol, quaternary ammonium salts such as Bardac®, polyethylenimines (such as Lupasol® from BASF) and zinc complexes thereof, silver and silver compounds, a cationic biocide including octyl decyl dimethyl ammonium chloride, dioctyl dimethyl ammonium chloride, didecyl dimethyl ammonium chloride, dispersant, cleaning polymer, glucan, or a mixture thereof.

The organic solvent may include an alcohol and/or a polyol. For example, the organic solvent may comprise ethanol, propanol, isopropanol, a sugar alcohol, a glycol, a glycol ether, or a combination thereof. The organic solvent may comprise polyethylene glycol, especially low molecular weight polyethylene glycols such as PEG 200 and PEG 400; diethylene glycol; glycerol; 1,2-propanediol; polypropylene glycol including dipropylene glycol and tripropylene glycol and low molecular weight polypropylene glycols such as PPG400; or a mixture thereof.

The enzyme may comprise, for example, protease, amylase, cellulase, mannanase, lipase, xyloglucanase, pectate lyase, nuclease enzyme, or a mixture thereof.

The composition may comprise one or more polymers. Examples are optionally modified carboxymethylcellulose, modified polyglucans, poly(vinyl-pyrrolidone), poly(ethylene glycol), poly(vinyl alcohol), poly(vinylpyridine-N-oxide), poly(vinylimidazole), polycarboxylates such as polyacrylates, maleic/acrylic acid copolymers and lauryl methacrylate/acrylic acid co-polymers.

The composition may comprise one or more amphiphilic cleaning polymers. Such polymers have balanced hydrophilic and hydrophobic properties such that they remove grease particles from fabrics and surfaces. Suitable amphiphilic alkoxylated grease cleaning polymers comprise a core structure and a plurality of alkoxylate groups attached to that core structure. These may comprise alkoxylated polyalkylenimines, especially ethoxylated polyethylene imines or polyethyleneimines having an inner polyethylene oxide block and an outer polypropylene oxide block. Typically, these may be incorporated into the compositions of the invention in amounts of from 0.005 to 10 wt %, generally from 0.5 to 8 wt %.

The composition may comprise a zwitterionic polyamine that is a modified hexamethylenediamine. The modification of the hexamethylenediamine includes: (1) one or two alkoxylation modifications per nitrogen atom of the hexamethylenediamine. The alkoxylation modification consisting of the replacement of a hydrogen atom on the nitrogen of the hexamethylenediamine by a (poly)alkoxylene chain having an average of about 1 to about 40 alkoxy moieties per modification, wherein the terminal alkoxy moiety of the alkoxylene chain is capped with hydrogen, a C1-C4 alkyl, sulfates, carbonates, or mixtures thereof; (2) a substitution of one C1-C4 alkyl moiety and one or two alkoxylation modifications per nitrogen atom of the hexamethylenediamine. The alkoxylation modification consisting of the replacement of a hydrogen atom by a (poly)alkoxylene chain having an average of about 1 to about 40 alkoxy moieties per modification wherein the terminal alkoxy moiety of the alkoxylene chain is capped with hydrogen, a C1-C4 alkyl or mixtures thereof; or (3) a combination thereof.

### Graft Polymers Based on Polyalkylene Oxide

The composition may comprise graft polymers which comprising polyalkylene oxide backbone (A) as a graft base and polymeric sidechains (B) grafted thereon. The polymeric sidechains (B) are obtainable by polymerization of at least one vinyl ester monomer. The polyalkylene oxide backbone (A) is obtainable by polymerization of at least one monomers selected from the group of ethylene oxide, 1,2-propylene oxide, 1,2-butylene oxide, 2,3-butylene oxide, 1,2-pentene oxide or 2,3-pentene oxide. Such graft polymers are known as effective soil suspension polymers for hydrophobic and hydrophilic stains, surfactant boosters, and sometimes as dye transfer inhibitors. Suitable graft polymers include amphiphilic graft co-polymer comprises polyethylene glycol backbone (A) as a graft base, and at least one pendant sidechains (B) selected from polyvinyl acetate, polyvinyl alcohol and mixtures thereof. A preferred graft polymer of this type is Sokalan HP22 available from BASF.

Suitable graft polymers are also described in WO2007/138053 as amphiphilic graft polymers based on water-soluble polyalkylene oxides (A) as a graft base and side chains formed by polymerization of a vinyl ester component (B), said polymers having an average of <one graft site per 50 alkylene oxide units and mean molar masses M of from 3 000 to 100 000. One specific preferred graft polymer of this type is polyvinyl acetate grafted polyethylene oxide copolymer having a polyethylene oxide as graft base and multiple polyvinyl acetate side chains. The molecular weight of the polyethylene oxide backbone is about 6000 and the weight ratio of the polyethylene oxide to polyvinyl acetate is about 40 to 60 and no more than 1 grafting point per 50 ethylene oxide units. The most preferred polymer of this type is available from BASF as Sokalan PG101.

Suitable graft polymer also include graft polymer comprising a block copolymer backbone (A) as a graft base, wherein said block copolymer backbone (A) is obtainable by polymerization of at least two monomers selected from the group of ethylene oxide, 1,2-propylene oxide, 1,2-butylene oxide, 2,3-butylene oxide, 1,2-pentene oxide or 2,3-pentene oxide, wherein the number (x) of individual blocks within the block copolymer backbone (A) is an integer, wherein x is from 2 to 10 and preferably 3 to 5, and (B) polymeric sidechains grafted onto the block copolymer backbone, wherein said polymeric sidechains (B) are obtainable by polymerization of at least one vinyl ester monomer. Suitable graft polymers of this type are described in WO2021/160795 and WO2021/160851, these polymers have improved biodegradation profiles.

Suitable graft polymer also include graft polymer comprising a polyalkylene oxide backbone (A) which has a number average molecular weight of from about 1000 to about 20,000 Daltons and is based on ethylene oxide, propylene oxide, or butylene oxide; and side chains derived from N-vinylpyrrolidone (B), and side chains derived from vinyl ester (C) derived from a saturated monocarboxylic acid containing from 1 to 6 carbon atoms and/or a methyl or ethyl ester of acrylic or methacrylic acid. Such graft polymers are described in WO2020005476 and can be used as dye transfer inhibitors.

The composition may comprise one or more soil release polymers. Examples include soil release polymers having a structure as defined by one of the following Formula (17), (18) or (19):

—[(OCHR₇—CHR₈)_{g}—O—OC—Ar—CO—]_{d} (17)

—[(OCHR₉—CHR₁₀)ₕ—O—OC-sAr—CO—]ₑ (18)

—[(OCHR₁₁—CHR₁₂)ᵢ—OR₁₃]_{f} (19)

wherein:
d, e and f are from 1 to 50;
g, h and i are from 1 to 200;
Ar is a 1,4-substituted phenylene;
sAr is 1,3-substituted phenylene substituted in position 5 with SO₃Me;
Me is Na, Li, K, Mg/2, Ca/2, Al/3, ammonium, mono-, di-, tri-, or tetraalkylammonium wherein the alkyl groups are C₁-C₁₈ alkyl or C₂-C₁₀ hydroxyalkyl, or mixtures thereof;
R₇, R₈, R₉, R₁₀, R₁₁ and R₁₂ are independently selected from H or C₁-C₁₈ n- or iso-alkyl; and
R₁₃ is a linear or branched C₁-C₁₈ alkyl, or a linear or branched C₂-C₃₀ alkenyl, or a cycloalkyl group with 5 to 9 carbon atoms, or a C₈-C₃₀ aryl group, or a C₆-C₃₀ arylalkyl group.

Suitable soil release polymers are polyester soil release polymers such as Repel-o-tex polymers, including Repel-o-tex SF, SF-2 and SRP6 supplied by Rhodia. Other suitable soil release polymers include Texcare polymers, including Texcare SRA100, SRA300, SRN100, SRN170, SRN240, SRN260, SRN300 and SRN325 supplied by Clariant. Other suitable soil release polymers are Marloquest polymers, such as Marloquest SL supplied by Sasol. Known polymeric soil release agents, hereinafter “SRA” or “SRA's”, may optionally be employed in the present detergent compositions. If utilized, SRA's will generally comprise from 0.01% to 10.0%, typically from 0.1% to 5%, preferably from 0.2% to 3.0% by weight, of the composition.

SRA's may include, for example, a variety of charged, e.g., anionic or even cationic (see U.S. Pat. No. 4,956,447), as well as noncharged monomer units and structures may be linear, branched or even star-shaped. Examples of SRAs are described in U.S. Pat. Nos. 4,968,451; 4,711,730; 4,721,580; 4,702,857; 4,877,896; 3,959,230; 3,893,929; 4,000,093; 5,415,807; 4,201,824; 4,240,918; 4,525,524; 4,201,824; 4,579,681; and 4,787,989.

The composition may comprise a carboxylate polymer, such as a maleate/acrylate random copolymer or polyacrylate homopolymer. Suitable carboxylate polymers include: polyacrylate homopolymers having a molecular weight of from 4,000 Da to 9,000 Da; maleate/acrylate random copolymers having a molecular weight of from 50,000 Da to 100,000 Da, or from 60,000 Da to 80,000 Da.

Alternatively, these materials may comprise polyacrylates having one ethoxy side-chain per every 7-8 acrylate units. The side-chains are of the formula —(CH₂CH₂O)ₘ(CH₂)ₙCH₃ wherein m is 2-3 and n is 6-12. The side-chains are ester-linked to the polyacrylate “backbone” to provide a “comb” polymer type structure. The molecular weight may vary, but is typically in the range of about 2000 to about 50,000. Such alkoxylated polycarboxylates may comprise from about 0.05% to about 10%, by weight, of the compositions herein.

Such carboxylate-based polymers may advantageously be utilized at levels from about 0.1% to about 7%, by weight, in the compositions herein. Suitable polymeric dispersing agents include carboxylate polymer such as a maleate/acrylate random copolymer or polyacrylate homopolymer. Preferably the carboxylate polymer is a polyacrylate homopolymer having a molecular weight of from 4,000 Daltons to 9,000 Daltons, or maleate/acrylate copolymer with a molecular weight 60,000 Daltons to 80,000 Daltons. Polymeric polycarboxylates and polyethylene glycols, may also be used. Polyalkylene glycol-based graft polymer may prepared from the polyalkylene glycol-based compound and the monomer material, wherein the monomer material includes the carboxyl group-containing monomer and the optional additional monomer(s). Optional additional monomers not classified as a carboxyl group-containing monomer include sulfonic acid group-containing monomers, amino group-containing monomers, allylamine monomers, quaternized allylamine monomers, N vinyl monomers, hydroxyl group-containing monomers, vinylaryl monomers, isobutylene monomers, vinyl acetate monomers, salts of any of these, derivatives of any of these, and mixtures thereof.

The composition may comprise alkoxylated polyamines. Such materials include but are not limited to ethoxylated polyethyleneimine, ethoxylated hexamethylene diamine, and sulfated versions thereof. Polypropoxylated derivatives are also included. A wide variety of amines and polyaklyeneimines may be alkoxylated to various degrees, and optionally further modified to provide the abovementioned benefits. A useful example is 600 g/mol polyethyleneimine core ethoxylated to 20 EO groups per NH. A preferred ethoxylated polyethyleneimine is PE-20 available from BASF.

Useful alkoxylated polyamine based polymers include the alkoxylated polyethylene imine type where said alkoxylated polyalkyleneimine has a polyalkyleneimine core with one or more side chains bonded to at least one nitrogen atom in the polyalkyleneimine core, wherein said alkoxylated polyalkyleneimine has an empirical Formula (20) of

(PEI)ⱼ-(EO)ₖ—R₁₄, (20)

wherein j is the average number-average molecular weight (MW_{PEI}) of the polyalkyleneimine core of the alkoxylated polyalkyleneimine and is in the range of from 100 to 100,000 Daltons, wherein k is the average degree of ethoxylation in said one or more side chains of the alkoxylated polyalkyleneimine and is in the range of from 5 to 40, and wherein R₁₄ is independently selected from the group consisting of hydrogen, C₁-C₄ alkyls, and combinations thereof.

Other suitable alkoxylated polyalkyleneimine include those wherein said alkoxylated polyalkyleneimine has a polyalkyleneimine core with one or more side chains bonded to at least one nitrogen atom in the polyalkyleneimine core, wherein the alkoxylated polyalkyleneimine has an empirical Formula (21) of

(PEI)ₒ-(EO)ₘ(PO)ₙ—R₁₅ or (PEI)ₒ—(PO)ₙ(EO)ₘ—R₁₅, (21)

wherein o is the average number-average molecular weight (MW_{PEI}) of the polyalkyleneimine core of the alkoxylated polyalkyleneimine and is in the range of from 100 to 100,000 Daltons, wherein m is the average degree of ethoxylation in said one or more side chains of the alkoxylated polyalkyleneimine which ranges from 10 to 50, wherein n is the average degree of propoxylation in said one or more side chains of the alkoxylated polyalkyleneimine which ranges from 1 to 50, and wherein R₁₅ is independently selected from the group consisting of hydrogen, C₁-C₄ alkyls, and combinations thereof.

Cellulosic polymers may be used according to the invention. Suitable cellulosic polymers are selected from alkyl cellulose, alkyl alkoxyalkyl cellulose, carboxyalkyl cellulose, alkyl carboxyalkyl cellulose, sulphoalkyl cellulose, more preferably selected from carboxymethyl cellulose, methyl cellulose, methyl hydroxyethyl cellulose, methyl carboxymethyl cellulose, and mixtures thereof. Suitable carboxymethyl celluloses have a degree of carboxymethyl substitution from 0.5 to 0.9 and a molecular weight from 100,000 Da to 300,000 Da. Suitable carboxymethyl celluloses have a degree of substitution greater than 0.65 and a degree of blockiness greater than 0.45, e.g. as described in WO09/154933.

The consumer products of the present invention may also include one or more cellulosic polymers including those selected from alkyl cellulose, alkylalkoxyalkyl cellulose, carboxyalkyl cellulose, alkyl carboxyalkyl cellulose. In one aspect, the cellulosic polymers are selected from the group comprising carboxymethyl cellulose, methyl cellulose, methyl hydroxyethyl cellulose, methyl carboxymethyl cellulose, and mixtures thereof. In one aspect, the carboxymethyl cellulose has a degree of carboxymethyl substitution from 0.5 to 0.9 and a molecular weight from 100,000 Da to 300,000 Da. Examples of carboxymethylcellulose polymers are Carboxymethyl cellulose commercially sold by CPKelko as Finnfix®GDA, hydrophobically modified carboxymethyl cellulose, for example the alkyl ketene dimer derivative of carboxymethylcellulose sold commercially by CPKelco as Finnfix®SH1, or the blocky carboxymethylcellulose sold commercially by CPKelco as Finnfix®V.

Cationic polymers may also be used according to the invention. Suitable cationic polymers will have cationic charge densities of at least 0.5 meq/gm, in another embodiment at least 0.9 meq/gm, in another embodiment at least 1.2 meq/gm, in yet another embodiment at least 1.5 meq/gm, but in one embodiment also less than 7 meq/gm, and in another embodiment less than 5 meq/gm, at the pH of intended use of the composition, which pH will generally range from pH 3 to pH 9, in one embodiment between pH 4 and pH 8. Herein, “cationic charge density” of a polymer refers to the ratio of the number of positive charges on the polymer to the molecular weight of the polymer. The average molecular weight of such suitable cationic polymers will generally be between 10,000 and 10 million, in one embodiment between 50,000 and 5 million, and in another embodiment between 100,000 and 3 million.

Suitable cationic polymers for use in the compositions herein may contain cationic nitrogen-containing moieties such as quaternary ammonium or cationic protonated amino moieties. Any anionic counterions may be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability or aesthetics. Nonlimiting examples of such counterions include halides (e.g., chloride, fluoride, bromide, iodide), sulfate and methylsulfate.

Nonlimiting examples of such polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)).

Especially useful cationic polymers which may be used include wherein said cationic polymer comprises a polymer selected from the group consisting of cationic celluloses, cationic guars, poly(acrylamide-co-diallyldimethylammonium chloride), poly(acrylamide-co-diallyldimethylammonium chloride-co-acrylic acid), poly(acrylamide-co-methacryloamidopropyl-pentamethyl-1,3-propylene-2-ol-ammonium dichloride), poly(acrylamide-co-N,N-dimethylaminoethyl acrylate) and its quaternized derivatives, poly(acrylamide-co-N,N-dimethylaminoethyl methacrylate) and its quaternized derivatives, poly(acrylamide-methacrylamidopropyltrimethyl ammonium chloride), poly(acrylamide-methacrylamidopropyltrimethyl ammonium chloride-co-acrylic acid), poly(diallyldimethyl ammonium chloride), poly(diallyldimethylammonium chloride-co-acrylic acid), poly(ethyl methacrylate-co-oleyl methacrylate-co-diethylaminoethyl methacrylate) and its quaternized derivatives, poly(ethyl methacrylate-co-dimethylaminoethyl methacrylate) and its quaternized derivatives, poly(hydroxpropylacrylate-co-methacrylamidopropyltrimethylammonium chloride) and its quaternized derivatives, poly(hydroxyethylacrylate-co-dimethyl aminoethyl methacrylate) and its quaternized derivatives, poly(methylacrylamide-co-dimethylaminoethyl acrylate) and its quaternized derivatives, poly(methacrylate-co-methacrylamidopropyltrimethyl ammonium chloride), poly(vinylformamide-co-acrylic acid-co-diallyldimethylammonium chloride), poly(vinylformamide-co-diallyldimethylammonium chloride), poly(vinylpyrrolidone-co-acrylamide-co-vinyl imidazole) and its quaternized derivatives, poly(vinylpyrrolidone-co-dimethylaminoethyl methacrylate) and its quaternized derivatives, poly(vinylpyrrolidone-co-methacrylamide-co-vinyl imidazole) and its quaternized derivatives, poly(vinylpyrrolidone-co-vinyl imidazole) and its quaternized derivatives, polyethyleneimine and including its quaternized derivatives, and mixtures thereof

Other suitable cationic polymers for use in the composition include polysaccharide polymers, cationic guar gum derivatives, quaternary nitrogen-containing cellulose ethers, synthetic polymers, copolymers of etherified cellulose, guar and starch. When used, the cationic polymers herein are either soluble in the composition or are soluble in a complex coacervate phase in the composition formed by the cationic polymer and the anionic, amphoteric and/or zwitterionic surfactant component described hereinbefore. Complex coacervates of the cationic polymer may also be formed with other charged materials in the composition.

Suitable cationic polymers are described in U.S. Pat. Nos. 3,962,418; 3,958,581; and U.S. Publication No. 2007/0207109A1.

The composition may comprise one or more dye transfer inhibiting agents. In one embodiment of the invention the inventors have surprisingly found that compositions comprising polymeric dye transfer inhibiting agents in addition to the specified dye give improved performance. This is surprising because these polymers prevent dye deposition. Suitable dye transfer inhibitors include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. Suitable examples include PVP—K15, PVP—K30, ChromaBond S-400, ChromaBond S-403E and Chromabond S-100 from Ashland Aqualon, and Sokalan HP165, Sokalan HP50, Sokalan HP53, Sokalan HP59, Sokalan® HP 56K, Sokalan® HP 66 from BASF. The dye control agent may be selected from (i) a sulfonated phenol/formaldehyde polymer; (ii) a urea derivative; (iii) polymers of ethylenically unsaturated monomers, where the polymers are molecularly imprinted with dye; (iv) fibers consisting of water-insoluble polyamide, wherein the fibers have an average diameter of not more than about 2 μm; (v) a polymer obtainable from polymerizing benzoxazine monomer compounds; and (vi) combinations thereof. Other suitable DTIs are as described in WO2012/004134. When present in a subject composition, the dye transfer inhibiting agents may be present at levels from about 0.0001% to about 10%, from about 0.01% to about 5% or even from about 0.1% to about 3% by weight of the composition.

Examples of water soluble polymers include but are not limited to polyvinyl alcohols (PVA), modified PVAs; polyvinyl pyrrolidone; PVA copolymers such as PVA/polyvinyl pyrrolidone and PVA/polyvinyl amine; partially hydrolyzed polyvinyl acetate; polyalkylene oxides such as polyethylene oxide; polyethylene glycols; acrylamide; acrylic acid; cellulose, alkyl cellulosics such as methyl cellulose, ethyl cellulose and propyl cellulose; cellulose ethers; cellulose esters; cellulose amides; polyvinyl acetates; polycarboxylic acids and salts; polyaminoacids or peptides; polyamides; polyacrylamide; copolymers of maleic/acrylic acids; polysaccharides including starch, modified starch; gelatin; alginates; xyloglucans, other hemicellulosic polysaccharides including xylan, glucuronoxylan, arabinoxylan, mannan, glucomannan and galactoglucomannan; and natural gums such as pectin, xanthan, and carrageenan, locus bean, arabic, tragacanth; and combinations thereof.

The composition may comprise a fabric shading agent. Suitable fabric shading agents include dyes, dye-clay conjugates, and pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof. Preferred dyes include alkoxylated azothiophenes, Solvent Violet 13, Acid Violet 50 and Direct Violet 9.

### Encapsulates

Microcapsules: Suitable capsules are typically formed by at least partially, preferably fully, surrounding a benefit agent with a wall material. Preferably, the capsule is a perfume capsule, wherein said benefit agent comprises one or more perfume raw materials. The capsule wall material may comprise: melamine, polyacrylamide, silicones, silica, polystyrene, polyurea, polyurethanes, polyacrylate based materials, polyacrylate esters based materials, gelatin, styrene malic anhydride, polyamides, aromatic alcohols, polyvinyl alcohol, resorcinol-based materials, poly-isocyanate-based materials, acetals (such as 1,3,5-triol-benzene-gluteraldehyde and 1,3,5-triol-benzene melamine), starch, cellulose acetate phthalate and mixtures thereof. Preferably, the capsule wall comprises melamine and/or a polyacrylate based material. The perfume capsule may be coated with a deposition aid, a cationic polymer, a non-ionic polymer, an anionic polymer, or mixtures thereof. Preferably, the perfume capsules have a volume weighted mean particle size from 0.1 microns to 100 microns, preferably from 0.5 microns to 60 microns. Especially where the composition comprises capsules having a shell formed at least partially from formaldehyde, the composition can additionally comprise one or more formaldehyde scavengers.

The encapsulates may have a volume weighted median encapsulate size from about 0.5 microns to about 100 microns, or even 10 to 100 microns, preferably from about 1 micron to about 60 microns, or even 10 microns to 50 microns, or even 20 microns to 45 microns, or alternatively 20 microns to 60 microns.

### Hindered Phenol Antioxidant

The liquid laundry detergent composition may contain at least one hindered phenol antioxidant, preferably in an amount sufficient to provide at least 25 ppb, more preferably at least 100 ppb, even more preferably at least 250 ppb, even more preferably at least 500 ppb, most preferably at least 1000 ppb, of an antioxidant concentration in the treatment liquor. Without wishing to be bound by theory, it is believed that hindered phenol antioxidants may help to improve malodor control performance of the liquid laundry detergent compositions, particularly in combination with ethyl gallate, methyl gallate, propyl gallate

As used herein, the term “hindered phenol” is used to refer to a compound comprising a phenol group with substituent(s) at a position ortho to at least one phenolic-OH group. Preferably, the hindered phenol antioxidant used in the present invention comprises at least one phenolic-OH group having: (a) at least one C3-C22 branched alkyl at a position ortho to said at least one phenolic —OH group; or (b) substitutes at each position ortho to said at least one phenolic-OH group, wherein said substitutes are independently selected from the group consisting of hydroxy, C1-C6 alkoxy, C1-C22 linear alkyl, and combinations thereof.

Examples of such hindered phenol antioxidants may include, but are not limited to: 2,6-bis(1-methylpropyl) phenol; 2,6-bis(1,1-dimethylethyl)-4-methyl-phenol (also known as hydroxy butylated toluene or “BHT”); 2-(1,1-dimethylethyl)-1,4-benzenediol; 2,4-bis(1,1-dimethylethyl)-phenol; 2,6-bis(1,1-dimethylethyl)-phenol; 3,5-bis(1,1-dimethylethyl)-4-hydroxybenzene propanoic acid, methyl ester; 2-(1,1-dimethylethyl)-4-methylphenol; 2-(1,1-dimethylethyl)-4,6-dimethyl-phenol; 3,5-bis(1,1-dimethylethyl)-4-hydroxybenzenepropanoic acid, 1,1′-[2,2-bis[[3-[3,5-bis(1,1-dimethylethyl)-4-hydroxyphenyl]-1-oxopropoxy]methyl]-1,3-propanediyl]ester; 3,5-bis(1,1-dimethylethyl)-4-hydroxybenzenepropanoic acid, octadecyl ester; 2,2′-methylenebis[6-(1,1-dimethylethyl)-4-methylphenol; 2-(1,1-dimethylethyl)-phenol; 2,4,6-tris(1,1-dimethylethyl)-phenol; 4,4′-methylenebis[2,6-bis(1,1-dimethylethyl)-phenol; 4,4′,4″-[(2,4,6-trimethyl-1,3,5-benzenetriyl)tris(methylene)]tris[2,6-bis(1,1-dimethylethyl)-phenol]; N,N′-1,6-hexanediylbis[3,5-bis(1,1-dimethylethyl)-4-hydroxybenzenepropanamide; 3,5-bis(1,1-dimethylethyl)-4-hydroxy benzoic acid, hexadecyl ester; P-[[3,5-bis(1,1-dimethylethyl)-4-hydroxyphenyl]methylphosphonic acid, diethyl ester; 1,3,5-tris[[3,5-bis(1,1-dimethylethyl)-4-hydroxyphenyl]methyl]-1,3,5-Triazine-2,4,6(1H,3H,5H)-trione; 3,5-bis(1,1-5 dimethylethyl)-4-hydroxybenzenepropanoic acid, 2-[3-[3,5-bis(1,1-dimethylethyl)-4-hydroxyphenyl]-1-oxopropyl]hydrazide; 3-(1,1-dimethyl ethyl)-4-hydroxy-5-methylbenzenepropanoic acid, 1,1′-[1,2-ethanediylbis(oxy-2,1-ethanediyl)]ester; 4-[(dimethylamino)methyl]-2,6-bis(1,1-dimethylethyl)phenol; 4-[[4,6-bis(octylthio)-1,3,5-triazin-2-yl]amino]-2,6-bis(1,1-dimethylethyl)phenol; 3,5-bis(1,1-dimethylethyl)-4-hydroxy benzene propanoic acid, 1,1′-(thiodi-2,1-ethanediyl)ester; 3,5-bis(1,1-dimethylethyl)-4-hydroxybenzoic acid, 2,4-bis(1,1-dimethylethyl)phenyl ester; 3,5-bis(1,1-dimethylethyl)-4-hydroxybenzenepropanoic acid, 1,1′-(1,6-hexanediyl)ester; 3-(1,1-dimethylethyl)-4-hydroxy-5-methylbenzenepropanoic acid, 1,1′-[2,4,8,10-tetraoxaspiro[5.5]undecane-3,9-diylbis(2,2-dimethyl-2,1-ethanediyl)]ester; 3-(1,1-dimethylethyl)-b-[3-(1,1-dimethylethyl)-4-hydroxy phenyl]-4-hydroxy-b-methylbenzenepropanoic acid, 1,1′-(1,2-ethanediyl)ester; 2-[[3,5-bis(1,1-dimethylethyl)-4-hydroxyphenyl]methyl]-2-butylpropanedioic acid, 1,3-bis(1,2,2,6,6-pentamethyl-4-piperidinyl)ester; 3,5-bis(1,1-dimethylethyl)-4-hydroxybenzenepropanoic acid, 1-[2-[3-[3,5-bis(1,1-dimethylethyl)-4-hydroxyphenyl]-1-oxopropoxy]ethyl]-2,2,6,6-tetramethyl-4-piperidinyl ester; 3,4-dihydro-2,5,7,8-tetramethyl-2-[(4R,8R)-4,8,12-trimethyltridecyl]-(2R)-2H-1-benzopyran-6-ol; 2,6-dimethylphenol; 2,3,5-trimethyl-1,4-benzenediol; 2,4,6-trimethylphenol; 2,3,6-trimethylphenol; 4,4′-(1-methylethylidene)-bis[2,6-dimethylphenol]; 1,3,5-tris[[4-(1,1-dimethylethyl)-3-hydroxy-2,6-dimethylphenyl]methyl]-1,3,5-triazine-2,4,6(1H,3H,5H)-trione; 4,4′-methylenebis[2,6-dimethylphenol]; and mixtures thereof.

Additional antioxidants may be employed. Examples of suitable additional antioxidants for use include, but are not limited to, the group consisting of α-, β-, γ-, δ-tocopherol, ethoxyquin, 2,2,4-trimethyl-1,2-dihydroquinoline, 2,6-di-tert-butyl hydroquinone, tert-butyl hydroxyanisole, lignosulphonic acid and salts thereof, and mixtures thereof. It is noted that ethoxyquin (1,2-dihydro-6-ethoxy-2,2,4-trimethylquinoline) is marketed under the name Raluquin™ by the company Raschig™. Other types of antioxidants that may be used in the composition are 6-hydroxy-2,5,7,8-tetramethylchroman-2-carboxylic acid (Trolox™) and 1,2-benzisothiazoline-3-one (Proxel GXL™). Antioxidants such as tocopherol sorbate, butylated hydroxyl benzoic acids and their salts, gallic acid and its alkyl esters, uric acid and its salts, sorbic acid and its salts, and dihydroxyfumaric acid and its salts may also be useful. Other useful antioxidants may include tannins, such as tannins selected from the group consisting of gallotannins, ellagitannins, complex tannins, condensed tannins, and combinations thereof.

Preferably, the hindered phenol antioxidant comprises at least one phenolic-OH group having at least one C3-C6 branched alkyl at a position ortho to said at least one phenolic-OH group. For example, the hindered phenol antioxidant may be 2,6-bis(1,1-dimethylethyl)-4-methyl-phenol (BHT).

More preferably, the hindered phenol antioxidant is an ester of 3,5-bis(1,1-dimethylethyl)-4-hydroxy-benzenepropanoic acid, and most preferably a C1-C22 linear alkyl ester of 3,5-bis(1,1-dimethylethyl)-4-hydroxy-benzenepropanoic acid. Commercially available C1-C22 linear alkyl esters of 3,5-bis(1,1-dimethylethyl)-4-hydroxy-benzenepropanoic acid include from Raschig USA (Texas, USA), which is a methyl ester of 3,5-bis(1,1-dimethylethyl)-4-hydroxy-benzenepropanoic acid, and Tinogard TS from BASF (Ludwigshafen, Germany), which is an octadecyl ester of 3,5-bis(1,1-dimethylethyl)-4-hydroxy-benzenepropanoic acid.

The hindered phenol antioxidants may also help to reduce yellowing that may be associated with amines, allowing the amines to be formulated at a relatively higher level. In this respect, it is desirable to use non-yellowing hindered phenol antioxidants, may be preferred. Antioxidants that form yellow by-products are undesirable because they may lead to perceptible negative attributes in the consumer experience (such as deposition of yellow by-products on fabric, for example). The skilled artisan is able to make informed decisions regarding the selection of antioxidants to employ. Further,

The above-described hindered phenol antioxidant may be present in the liquid laundry detergent composition of the present invention in an amount ranging from about 0.001 wt % to about 5 wt %, preferably from about 0.005 wt % to about 2 wt %, more preferably from about 0.01 wt % to about 1 wt %, most preferably from about 0.02 wt % to about 0.5 wt %.

In a particularly preferred embodiment, the liquid laundry detergent composition contains from about 0.02 wt % to about 0.5 wt % of a C1-C22 linear alkyl ester of 3,5-bis(1,1-dimethylethyl)-4-hydroxy-benzenepropanoic acid.

### Oligoamines

Non-limiting examples of amines include, but are not limited to, etheramines, cyclic amines, polyamines, oligoamines (e.g., triamines, diamines, pentamines, tetraamines), or combinations thereof. The compositions described herein may comprise an amine selected from the group consisting of oligoamines, etheramines, cyclic amines, and combinations thereof. In some aspects, the amine is not an alkanolamine. In some aspects, the amine is not a polyalkyleneimine.

Examples of suitable oligoamines include Preferably the composition comprises oligoamines. Suitable oligoamines according to the present disclosure may include diethylenetriamine (DETA), 4-methyl diethylenetriamine (4-MeDETA), dipropylenetriamine (DPTA), 5-methyl dipropylenetriamine (5-MeDPTA), triethylenetetraamine (TETA), 4-methyl triethylenetetraamine (4-MeTETA), 4,7-dimethyl triethylenetetraamine (4,7-Me2TETA), 1,1,4,7,7-pentamethyl diethylenetriamine (M5-DETA), tripropylenetetraamine (TPTA), tetraethylenepentaamine (TEPA), tetrapropylenepentaamine (TPPA), pentaethylenehexaamine (PEHA), pentapropylenehexaamine (PPHA), hexaethyleneheptaamine (HEHA), hexapropyleneheptaamine (HPHA), N,N′-Bis(3-aminopropyl)ethylenediamine, 1,1,4,7,7-pentamethyl diethylenetriamine (M5-DETA), dipropylenetriamine (DPTA) or mixtures thereof most preferably diethylenetriamine (DETA). DETA may be preferred due to its low molecular weight and/or relatively low cost to produce.

The oligoamines of the present disclosure may have a molecular weight of between about 100 to about 1200 Da, or from about 100 to about 900 Da, or from about 100 to about 600 Da, or from about 100 to about 400 Da, preferably between about 100 Da and about 250 Da, most preferably between about 100 Da and about 175 Da, or even between about 100 Da and about 150 Da. For purposes of the present disclosure, the molecular weight is determined using the free base form of the oligoamine.

### Etheramines:

The cleaning compositions described herein may contain an etheramine. The cleaning compositions may contain from about 0.1% to about 10%, or from about 0.2% to about 5%, or from about 0.5% to about 4%, by weight of the composition, of an etheramine.

The etheramines of the present disclosure may have a weight average molecular weight of less than about grams/mole 1000 grams/mole, or from about 100 to about 800 grams/mole, or from about 200 to about 450 grams/mole, or from about 290 to about 1000 grams/mole, or from about 290 to about 900 grams/mole, or from about 300 to about 700 grams/mole, or from about 300 to about 450 grams/mole. The etheramines of the present invention may have a weight average molecular weight of from about 150, or from about 200, or from about 350, or from about 500 grams/mole, to about 1000, or to about 900, or to about 800 grams/mole.

A unit dose may be in the form of a fibrous water-soluble product. A fibrous water-soluble product can include one or more layers. These layers may be superposed upon one another. The layers may lay directly upon one another, have particles in between the layers, or combination thereof.

The fibrous water-soluble unit dose article may comprise of 50% or greater of bio-based materials, such as for example between 50% and 95% bio-based. Some of the individual components of the fibrous water-soluble unit dose article may be fully bio-based to create an article that has a total bio-based content of greater than 50%.

These fibrous water-soluble unit dose articles can be dissolved under various wash conditions, e.g., low temperature, low water and/or short wash cycles or cycles where consumers have been overloading the machine, especially with items having high water absorption capacities, while providing sufficient delivery of active agents for the intended effect on the target consumer substrates (with similar performance as today's liquid products).

The surface of the fibrous water-soluble unit dose article may comprise a printed area. The printed area may cover between about 10% and about 100% of the surface of the article. The area of print may comprise inks, pigments, dyes, bluing agents or mixtures thereof. The area of print may be opaque, translucent or transparent. The area of print may comprise a single color or multiple colors. The printed area maybe on more than one side of the article and contain instructional text, graphics, etc., The surface of the water-soluble unit dose article may comprise an aversive agent, for example a bittering agent. Suitable bittering agents include, but are not limited to, naringin, sucrose octacetate, quinine hydrochloride, denatonium benzoate, or mixtures thereof. Any suitable level of aversive agent may be used. Suitable levels include, but are not limited to, 1 to 5000 ppm, or even 100 to 2500 ppm, or even 250 to 2000 ppm.

The fibrous water-soluble unit dose articles may exhibit a thickness of greater than 0.01 mm and/or greater than 0.05 mm and/or greater than 0.1 mm and/or to about 100 mm and/or to about 50 mm and/or to about 20 mm and/or to about 10 mm and/or to about 5 mm and/or to about 2 mm and/or to about 0.5 mm and/or to about 0.3 mm.

The fibrous water-soluble unit dose articles may have basis weights of from about 500 grams/m² to about 5,000 grams/m², or from about 1,000 grams/m² to about 4,000 grams/m², or from about 1,500 grams/m² to about 3,500 grams/m², or from about 2,000 grams/m² to about 3,000 grams/m², or any combination thereof.

The fibrous water-soluble unit dose article may exhibit different regions, such as different regions of basis weight, density, caliper, and/or wetting characteristics. The fibrous water-soluble unit dose article may be compressed at the point of edge sealing. The fibrous water-soluble unit dose article may comprise texture on one or more of its surfaces. A surface of the fibrous water-soluble unit dose article may comprise a pattern, such as a non-random, repeating pattern. The fibrous water-soluble unit dose article may comprise apertures. The fibrous water-soluble unit dose article may comprise a fibrous structure having discrete regions of fibrous elements that differ from other regions of fibrous elements in the structure. The fibrous water-soluble unit dose article may be used as is or it may be coated with one or more active agents.

The fibrous water-soluble unit dose article may comprise one or more plies. The fibrous water-soluble unit dose article may comprise at least two and/or at least three and/or at least four and/or at least five plies. The fibrous plies can be fibrous structures. Each ply may comprise one or more layers, for example one or more fibrous element layers, one or more particle layers, and/or one or more fibrous element/particle mixture layers. The layer(s) may be sealed. In particular, particle layers and fibrous element/particle mixture layers may be sealed, such that the particles do not leak out. The water-soluble unit dose articles may comprise multiple plies, where each ply comprises two layers, where one layer is a fibrous element layer and one layer is a fibrous element/particle mixture layer, and where the multiple plies are sealed (e.g., at the edges) together. Sealing may inhibit the leakage of particles as well as help the unit dose article maintain its original structure. However, upon addition of the water-soluble unit dose article to water, the unit dose article dissolves and releases the particles into the wash liquor.

The fibrous water-soluble unit dose may be in the form of any three-dimensional structure. The fibrous water-soluble unit dose article can be perforated. The article can also be cut or shaped into various sizes for different intended uses. For example, the water-soluble unit dose may be in the form of a square, a rounded square, a kite, a rectangle, a triangle, a circle, an ellipse, and mixtures thereof.

The fibrous water-soluble unit dose may comprise less than 10 ingredients. The water-soluble unit dose may comprise between 3 and 9 ingredients, such as, for example, 4 ingredients, 5 ingredients, 6 ingredients, 7 ingredients, or 8 ingredients.

The fibrous water-soluble unit dose articles disclosed herein comprise a water-soluble fibrous structure and one or more particles. The fibrous water-soluble fibrous structure may comprise a plurality of fibrous elements, for example a plurality of filaments. The one or more particles, for example one or more active agent-containing particles, may be distributed throughout the structure. The fibrous water-soluble unit dose article may comprise a plurality of two or more and/or three or more fibrous elements that are inter-entangled or otherwise associated with one another to form a fibrous structure and one or more particles, which may be distributed throughout the fibrous structure.

The fibrous water-soluble unit dose article may comprise a water-soluble fibrous structure. The water-soluble fibrous structure may comprise two or more different fibrous elements. Non-limiting examples of differences in the fibrous elements may be physical differences, such as differences in diameter, length, texture, shape, rigidness, elasticity, and the like; chemical differences, such as crosslinking level, solubility, melting point, Tg, active agent, filament-forming material, color, level of active agent, basis weight, level of filament-forming material, presence of any coating on fibrous element, biodegradable or not, hydrophobic or not, contact angle, and the like; differences in whether the fibrous element loses its physical structure when the fibrous element is exposed to conditions of intended use; differences in whether the fibrous element's morphology changes when the fibrous element is exposed to conditions of intended use; and differences in rate at which the fibrous element releases one or more of its active agents when the fibrous element is exposed to conditions of intended use. Two or more fibrous elements within the fibrous structure may comprise different active agents. This may be the case where the different active agents may be incompatible with one another, for example an anionic surfactant and a cationic polymer. When using different fibrous elements, the resulting structure may exhibit different wetting, imbibition, and solubility characteristics.

### Fibrous Structure

Fibrous structures comprise one or more fibrous elements. The fibrous elements can be associated with one another to form a structure. Fibrous structures can include particles within and or on the structure. Fibrous structures can be homogeneous, layered, unitary, zoned, or as otherwise desired, with different active agents defining the various aforesaid portions.

A fibrous structure can comprise one or more layers, the layers together forming a ply.

### Fibrous Elements

The fibrous elements may be water-soluble. The fibrous elements may comprise one or more filament-forming materials and/or one or more active agents, such as a surfactant described above. The one or more active agents may be releasable from the fibrous element, such as when the fibrous element and/or fibrous structure comprising the fibrous element is exposed to conditions of intended use.

The fibrous elements may be spun from a filament-forming composition, also referred to as fibrous element-forming compositions, via suitable spinning process operations, such as meltblowing, spunbonding, electro-spinning, and/or rotary spinning.

“Filament-forming composition” and/or “fibrous element-forming composition” as used herein means a composition that is suitable for making a fibrous element such as by meltblowing and/or spunbonding. The filament-forming composition comprises one or more filament-forming materials that exhibit properties that make them suitable for spinning into a fibrous element. The filament-forming material may comprise a polymer. In addition to one or more filament-forming materials, the filament-forming composition may comprise one or more active agents, for example, a surfactant. In addition, the filament-forming composition may comprise one or more polar solvents, such as water, into which one or more, for example all, of the filament-forming materials and/or one or more, for example all, of the active agents are dissolved and/or dispersed prior to spinning a fibrous element, such as a filament from the filament-forming composition.

The filament-forming composition may comprise two or more different filament-forming materials. Thus, the fibrous elements may be monocomponent (one type of filament-forming material) and/or multicomponent, such as bicomponent. The two or more different filament-forming materials may be randomly combined to form a fibrous element. The two or more different filament-forming materials may be orderly combined to form a fibrous element, such as a core and sheath bicomponent fibrous element, which is not considered a random mixture of different filament-forming materials for purposes of the present disclosure. Bicomponent fibrous elements may be in any form, such as side-by-side, core and sheath, islands-in-the-sea and the like.

The fibrous elements may each contain at least one filament-forming material. In addition, the fibrous element may contain one or more active agents. These active agents can be any of the materials mentioned above for inclusion in a detergent composition.

The fibrous element may comprise at least about 5%, and/or at least about 10%, and/or at least about 15%, and/or at least about 20%, and/or less than about 80%, and/or less than about 75%, and/or less than about 65%, and/or less than about 60%, and/or less than about 55%, and/or less than about 50%, and/or less than about 45%, and/or less than about 40%, and/or less than about 35%, and/or less than about 30%, and/or less than about 25% by weight on a dry fibrous element basis and/or dry fibrous structure basis of the filament-forming material and greater than about 20%, and/or at least about 35%, and/or at least about 40%, and/or at least about 45%, and/or at least about 50%, and/or at least about 55%, and/or at least about 60%, and/or at least about 65%, and/or at least about 70%, and/or less than about 95%, and/or less than about 90%, and/or less than about 85%, and/or less than about 80%, and/or less than about 75% by weight on a dry fibrous element basis and/or dry fibrous structure basis of an active agent, preferably surfactant. The fibrous element may comprise greater than about 80% by weight on a dry fibrous element basis and/or dry fibrous structure basis of surfactant.

Preferably, each fibrous element may be characterized by a sufficiently high total surfactant content, e.g., at least about 30%, or at least about 40%, or at least about 50%, or at least about 60%, or at least about 70%, by weight on a dry fibrous element basis and/or dry fibrous structure basis of the first surfactant.

The total level of filament-forming materials present in the fibrous element may be from about 5% to less than about 80% by weight on a dry fibrous element basis and/or dry fibrous structure basis and the total level of surfactant present in the fibrous element may be greater than about 20% to about 95% by weight on a dry fibrous element basis and/or dry fibrous structure basis.

In general, fibrous elements are elongated particulates having a length greatly exceeding average diameter, e.g., a length to average diameter ratio of at least about 10. A fibrous element may be a filament or a fiber. Filaments are relatively longer than fibers. A filament may have a length of greater than or equal to about 5.08 cm (2 in.), and/or greater than or equal to about 7.62 cm (3 in.), and/or greater than or equal to about 10.16 cm (4 in.), and/or greater than or equal to about 15.24 cm (6 in.). A fiber may have a length of less than about 5.08 cm (2 in.), and/or less than about 3.81 cm (1.5 in.), and/or less than about 2.54 cm (1 in.).

The one or more filament-forming materials and active agents may be present in the fibrous element at a weight ratio of total level of filament-forming materials to active agents of about 2.0 or less, and/or about 1.85 or less, and/or less than about 1.7, and/or less than about 1.6, and/or less than about 1.5, and/or less than about 1.3, and/or less than about 1.2, and/or less than about 1, and/or less than about 0.7, and/or less than about 0.5, and/or less than about 0.4, and/or less than about 0.3, and/or greater than about 0.1, and/or greater than about 0.15, and/or greater than about 0.2. The one or more filament-forming materials and active agents may be present in the fibrous element at a weight ratio of total level of filament-forming materials to active agents of about 0.2 to about 0.7.

The fibrous element may comprise from about 10% to less than about 80% by weight on a dry fibrous element basis and/or dry fibrous structure basis of a filament-forming material, such as polyvinyl alcohol polymer, starch polymer, and/or carboxymethylcellulose polymer, and greater than about 20% to about 90% by weight on a dry fibrous element basis and/or dry fibrous structure basis of an active agent, such as surfactant. The fibrous element may further comprise a plasticizer, such as glycerin, and/or additional pH adjusting agents, such as citric acid. The fibrous element may have a weight ratio of filament-forming material to active agent of about 2.0 or less. The filament-forming material may be selected from the group consisting of polyvinyl alcohol, starch, carboxymethylcellulose, polyethylene oxide, and other suitable polymers, especially hydroxyl-containing polymers and their derivatives. The filament-forming material may range in weight average molecular weight from about 100,000 g/mol to about 3,000,000 g/mol. It is believed that in this range, the filament-forming material may provide extensional rheology, without being so elastic that fiber attenuation is inhibited in the fiber-making process.

The one or more active agents may be releasable and/or released when the fibrous element and/or fibrous structure comprising the fibrous element is exposed to conditions of intended use. The one or more active agents in the fibrous element may be selected from the group consisting of surfactants, organic polymeric compounds, and mixtures thereof.

The fibrous elements may exhibit a diameter of less than about 300 μm, and/or less than about 75 μm, and/or less than about 50 μm, and/or less than about 25 μm, and/or less than about 10 μm, and/or less than about 5 μm, and/or less than about 1 μm. The fibrous elements may exhibit a diameter of greater than about 1 μm as measured according to the Diameter Test Method described herein. The diameter of a fibrous element may be used to control the rate of release of one or more active agents present in the fibrous element and/or the rate of loss and/or altering of the fibrous element's physical structure.

The active agents in a fibrous water soluble product may also be in the form of particles. Particles may be incorporated into a fibrous water soluble product at a level of about 0.1 g to about 70 g. The type of particles utilized can be any that are compatible with the manufacturing system.

The particles may be a powder, granule, agglomerate, encapsulate, microcapsule, and/or prill. The particles may be made using a number of well-known methods in the art, such as spray-drying, agglomeration, extrusion, prilling, encapsulation, pastillation, and combinations thereof. The shape of the particles can be in the form of spheres, rods, plates, tubes, squares, rectangles, discs, stars, fibers or have regular or irregular random forms. The particles may have a D50 particle size of from about 100 μm to about 1600 μm.

The particles may include a mixture of chemically different actives, such as: surfactant particles, including surfactant agglomerates, surfactant extrudates, surfactant needles, surfactant noodles, surfactant flakes; phosphate particles; zeolite particles; silicate salt particles, especially sodium silicate particles; carbonate salt particles, especially sodium carbonate particles; polymer particles such as carboxylate polymer particles, cellulosic polymer particles, starch particles, polyester particles, polyamine particles, terephthalate polymer particles, polyethylene glycol particles; aesthetic particles such as colored noodles, needles, lamellae particles and ring particles; enzyme particles such as protease granulates, amylase granulates, lipase granulates, cellulase granulates, mannanase granulates, pectate lyase granulates, xyloglucanase granulates, bleaching enzyme granulates and co-granulates of any of these enzymes, these enzyme granulates may comprise sodium sulphate; bleach particles, such as percarbonate particles, especially coated percarbonate particles, such as percarbonate coated with carbonate salt, sulphate salt, silicate salt, borosilicate salt, or any combination thereof, perborate particles, bleach activator particles such as tetra acetyl ethylene diamine particles and/or alkyl oxybenzene sulphonate particles, bleach catalyst particles such as transition metal catalyst particles, and/or isoquinolinium bleach catalyst particles, pre-formed peracid particles, especially coated pre-formed peracid particles; filler particles such as sulphate salt particles and chloride particles; clay particles such as montmorillonite particles and particles of clay and silicone; flocculant particles such as polyethylene oxide particles; wax particles such as wax agglomerates; silicone particles, brightener particles; dye transfer inhibition particles; dye fixative particles; perfume particles such as perfume microcapsules and starch encapsulated perfume accord particles, or pro-perfume particles such as Schiff base reaction product particles; hueing dye particles; chelant particles such as chelant agglomerates; and any combination thereof.

### Combinations

Specifically contemplated combinations of the disclosure are herein described in the following lettered paragraphs. These combinations are intended to be illustrative in nature and are not intended to be limiting.

A. A detergent composition comprising at least one component, according to Formula 7:

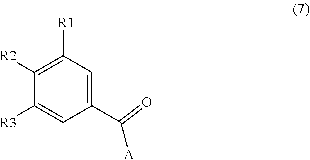

in which R₁₋₃ is an OH or an H and at least 2 adjacent R's are OH; and A is an OR₄ or N(R₄)₂ or N(R₄)(H) or NH₂, where each R₄ is independently a linear or branched C₁₋₁₈ carbon chain; and wherein the pH of the detergent composition is between about 6 and about 9.

B. A detergent composition according to Paragraph A, wherein the at least one component according to Formula 7 is ethyl gallate, methyl gallate, propyl gallate, or a combination thereof.

C. A detergent composition according to any of Paragraphs A-B, wherein the at least one component according to Formula 7 is ethyl gallate.

D. The detergent composition according to any of Paragraphs A-C, wherein the at least one component has a pKa of less than about 9.5

E. The detergent composition according to any of Paragraphs A-D, wherein the at least one component is present in an amount of greater than 0 to about 10% by weight of the detergent composition.

F. The detergent composition according to any of Paragraphs A-E, further comprising a surfactant comprising an anionic surfactant, a nonionic surfactant, or a combination thereof.

G. The detergent composition according Paragraph F, wherein the surfactant is an anionic surfactant comprising at least one of, alkylalkoxylated sulfate, sodium lauryl sulfate, linear alkyl benzene sulfonic acid, branched 2-alkyl primary alkyl alcohol sulfate, alkyl sulphate, or a combination thereof.

H. The detergent composition according to any of Paragraphs A-G, wherein the composition is a liquid composition.

I. The detergent composition according to any of Paragraphs A-H, and dye transfer inhibition (DTI) polymer wherein the dye transfer inhibition polymer is a graft copolymer, the graft copolymer comprising:
a. polyalkylene oxide which has a number average molecular weight of from about 1000 to about 20,000 Daltons and is based on ethylene oxide, propylene oxide, or butylene oxide;
b. N-vinylpyrrolidone; and
c. vinyl ester derived from a saturated monocarboxylic acid containing from 1 to 6 carbon atoms and/or a methyl or ethyl ester of acrylic or methacrylic acid;
wherein the weight ratio of (a) to (b) is from about 1:0.1 to about 1:2

J. The detergent composition according to any of Paragraphs A-I, and an antioxidant wherein the antioxidant is 3,5-bis(1,1-dimethylethyl)-4-hydroxy-benzenepropanoic acid, methyl ester.

K. A single fibrous water-soluble unit dose detergent comprising the detergent composition according to any of Paragraphs A-J.

L. A method for treating a stain on a fabric, comprising:
washing the fabric in a wash liquor comprising a detergent composition comprising at least one component, according to Formula 7:

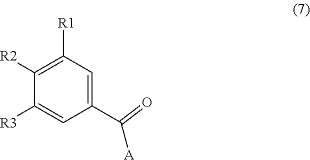

in which:
   R₁₋₃ is an OH or an H and at least 2 adjacent R's are OH; and
A is a OR₄ or N(R₄)₂ or N(R₄)(H) or NH₂, where each R₄ is independently a linear or branched C1-18 carbon chain.

M. The method according to Paragraph L, wherein the at least one component according to Formula 7 is ethyl gallate, methyl gallate, propyl gallate, or a combination thereof.

N. The method according to any of Paragraphs L-M, wherein the at least one component has a pKa of less than about 9.5.

0. The method according to any of Paragraphs L-N, wherein the at least one component is present in an amount of from greater than 0 to about 10% by weight of the detergent composition.

P. The method according to any one of Paragraphs L-O, further comprising maintaining the pH of the wash liquor at less than about 9, preferably from about 6.5 to about 8.5, most preferably from about 7.0 to about 8.0.

Q. The method according to any to Paragraphs L-P, wherein the stain comprises beverage, fruit, vegetable, or a combination thereof.

R. The method according to any of Paragraphs L-Q, wherein the detergent composition is in the form of a liquid, gel, powder, bead, granule, fiber, or any combination thereof.

S. The method according to any of Paragraphs L-R, wherein the detergent composition is in the form of a single fibrous water-soluble unit dose.

T. A method for pre-treating a stain on a fabric, comprising:
applying a detergent composition to the fabric, the detergent composition comprising at least one component, according Formula 7:

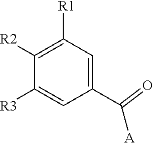

in which:
   R₁₋₃ is an OH or an H and at least 2 adjacent R's are OH; and
   A is a OR₄ or N(R₄)₂ or N(R₄)(H) or NH₂, where each R₄ is independently a linear or branched C1-18 carbon chain.

U. The method according to Paragraph T, wherein the at least one component according to Formula 7 is ethyl gallate, methyl gallate, propyl gallate, or a combination thereof.

V. A method for reducing dye transfer to a fabric, comprising:
washing the fabric in a wash liquor comprising a detergent composition comprising at least one component, according Formula 7:

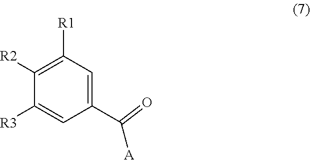

in which:
   R₁₋₃ is an OH or an H and at least 2 adjacent R's are OH; and
   A is a OR₄ or N(R₄)₂ or N(R₄)(H) or NH₂, where each R₄ is independently a linear or branched C1-18 carbon chain.

W. The method according to Paragraph V, wherein the at least one component according to Formula 7 is ethyl gallate, methyl gallate, propyl gallate, or a combination thereof.

X. The method according to any of Paragraphs V-W, wherein the at least one component has a pKa of about 9.5 or less.

Y. The method according to any one of Paragraphs V-X, wherein the at least one component is present in an amount of greater than 0 to about 10% by weight of the detergent composition.

Z. The method according to any one of Paragraphs V-Y, further comprising maintaining the pH of the wash liquor at less than about 9, preferably from about 6.5 to about 8.5, most preferably from about 7.0 to about 8.5.

AA. The method according to any one of Paragraphs V-Z, wherein the detergent composition is in the form or a liquid, gel, powder, bead, granule, fiber, or any combination thereof.

BB. The method according to Paragraphs V-AA, wherein the single unit dose is a water-soluble fibrous article.

CC. The method according to any one of Paragraphs V—BB, wherein the fabric comprises nylon, spandex, polyester, or a combination thereof.

DD. The method according to any one of Paragraphs V—CC, wherein the dye comprises a blue dye.

EE. Use of ethyl gallate, methyl gallate, propyl gallate, or a combination thereof, to enhance a stain removal ability of a detergent composition on a fabric.

FF. The use of ethyl gallate, methyl gallate, propyl gallate, or a combination thereof, wherein the stain comprises beverage, fruit, vegetable, or a combination thereof.

GG. The use of ethyl gallate, methyl gallate, propyl gallate, or a combination thereof, wherein the fabric comprises clothing.

HH. Use of ethyl gallate, methyl gallate, propyl gallate, or a combination thereof, to reduce a transfer of dye from a wash liquor to a fabric while laundering the fabric.

II. The use of ethyl gallate, methyl gallate, propyl gallate, or a combination thereof, wherein the dye comprises a blue dye.

JJ. The use of a ethyl gallate, methyl gallate, propyl gallate, or a combination thereof, wherein the fabric comprise nylon spandex, polyester spandex, or a combination thereof.

### Test Methods

### Stain Removal Index Calculation Method

Image analysis may be used to compare each stain to an unstained fabric control. Software may be used to convert images taken into standard colorimetric values and to compare these to standards based on the commonly used Macbeth Color Rendition Chart, assigning each stain a colorimetric value (Stain Level). The CIELAB color space, also referred to as L*a*b*, is a color space defined by the International Commission on Illumination (abbreviated CIE) in 1976.[a] It expresses color as three values: L* for perceptual lightness and a* and b* for the four unique colors of human vision: red, green, blue and yellow. The lightness value, L* defines black at 0 and white at 100. The a* axis is relative to the green-magenta opponent colors, with negative values toward green and positive values toward magenta. The b* axis represents the blue-yellow opponents, with negative numbers toward blue and positive toward yellow. In the following examples, various fabrics were analyzed using commercially available image analysis software for L*a*b* values. SRI values were then calculated from the L*a*b* values using the formula shown. The higher the SRI, the better the stain removal.

$$\% Stain Removal Index ( SRI ) = \frac{Δ Eb - Δ Ea}{Δ Eb} \times 100$$

$$in which ,$$

$$Δ {E}_{b} = √ ( {( {L}_{c} - {L}_{b} b )}^{2} + {( {a}_{c} - {a}_{b} )}^{2} + {( {b}_{c} - {b}_{b} )}^{2} )$$

$$Δ {E}_{a} = √ ( {( {L}_{c} - {L}_{a} )}^{2} + {( {a}_{c} - {a}_{a} )}^{2} + {( {b}_{c} - {b}_{a} )}^{2} )$$

Subscript ‘b’ denotes data for the stain before washing
Subscript ‘a’ denotes data for the stain after washing
Subscript ‘c’ denotes data for the unstained fabric

Thus, L*a*b* values are taken of the unstained fabric, of the stained fabric before washing and of the stained fabric after washing.

Two liquid detergent compositions were made and tested as detailed herein below. The composition for each is provided in Table 2. Table 2 provides amounts in ppm of the given component active in the wash solution.

TABLE 2

| | ppm Active in | |
|---|---|---|
| | wash solution | |
| | for Composition | |
| Component | C2-1 | C2-2 |
| Deionised Water | 1273 | 1273 |
| HLAS | 327 | 327 |
| C14-C15 ALCOHOL | 263 | 263 |
| ETHOXYLATE AE6.8-8.0 | | |
| C10-C16 ALCOHOL | 27 | 27 |
| ETHOXYLATE AE9 | | |
| PEI600 EO24 PO16 | 36 | 36 |
| (Modified Polyethylene | | |
| Imine supplied by BASF) | | |
| CAUSTIC SODA LOW | 33 | 33 |
| IRON | | |
| MONOETHANOLAMINE | 33 | 33 |
| LOW IRON | | |
| Amine Oxide | 15 | 15 |
| CITRIC ACID SOLUTION | 14 | 14 |
| POLYETHYLENEIMINE | 13 | 13 |
| ETHOXYLATE PEI600 E20. | | |
| (PE20) (Modified | | |
| Polyethylene Imine supplied | | |
| by BASF) | | |
| Alkyl Ethoxy Sulfate C24E3S | 13 | 13 |
| Tetrasodium Glutamate | 0 | 29.0 |
| Diacetate | | |
| C12-16 Alcohol Ethoxylate | 3.3 | 3.3 |
| AE9 | | |
| SODIUM CUMENE | 2.4 | 2.4 |
| SULFONATE | | |
| CALCIUM FORMATE | 2.2 | 2.2 |
| AF8017 Antifoam | 2.1 | 2.1 |
| (Supplied by Dow) | | |
| Protease | 2.0 | 2.0 |
| HYDROGENATED | 1.7 | 1.7 |
| CASTOR OIL. | | |
| Methyl 3-(3,5-di-tert-butyl-4- | 0.85 | 0.85 |
| hydroxyphenyl) propionate | | |
| Amylase | 0.45 | 0.456 |
| Mannanase | 0.091 | 0.091 |
| Xyloglucanase | 0.071 | 0.071 |
| Processing Aids, Minors, | 141 | 141 |
| Aesthetic Dyes and Perfumes | | |
| Total ppm | 2210 | 2239 |

The following method was used to test the ability of the example compositions to remove stains following treatment prior to the wash process. With reference to Table 3, Each of the compositions were added separately into pots of a tergotometer. The volume of each pot was 1 L. The wash temperature was set to 25° C. Throughout the procedure, 21 gpg water was used. The products were agitated for 1 minute (400 rpm) before addition of fabrics (two internal replicates of each stain, 11×6 cm² SBL2004 (supplied by WFK) and additional 6 cm² knitted cotton ballast squares to make the total fabric weight up to 60 g). Once the fabrics were added, the wash solution was agitated for 12 minutes (208 rpm). The wash solutions were then drained, and the fabrics were subjected to a 5-minute rinse step before being drained and spun dry. This procedure was repeated a further three times to give a total of four external replicates. After the wash, the stain fabrics were dried for 30 minutes in a tumble dryer.

TABLE 3

| | | pH | Final |
|---|---|---|---|
| Treatment | Treatment Composition | adjustment | pH |
| 2A | 2.21 g Composition C2-1 | 0.034 ml HCl | 7 |
| 2B | 2.23 g Composition C2-2 | 0.034 ml HCl | 7 |
| 2C | 2.21 g Composition C2-1 | | 8.5 |
| 2D | 2.23 g Composition C2-2 | | 8.5 |
| 2E | 2.21 g Composition C2-1 | 0.59 ml NaOH | 10 |
| 2F | 2.23 g Composition C2-2 | 0.59 ml NaOH | 10 |

Table 4 summarizes stain removal testing for each treatment.

TABLE 4

| | Tea | |
|---|---|---|
| | | Delta vs nil |
| | | Chelant |
| | | reference at |
| Treatment | SRI | same pH |
| 2A | 15.5 | |
| 2B | 19.0 | +3.5 |
| 2C | 5.6 | |
| 2D | 10.0 | +4.4 |
| 2E | −15.5 | |
| 2F | −15.4 | −0.1 |

As can be seen from Table 4, treatment compositions containing Ethyl Gallate, drive improved stain removal at pH<10

### Malodor Reduction Performance:

### A. Preparation of Malodor Core and Dosing Solutions

The malodor core solution was prepared according to Table 1 and the dosing solution according to Table 2. The solutions were made in glass jars with a Teflon-lined cap. Artificial body soil was commercially available by Accurate Product Development (APD) in Cincinnati, OH.

The malodor core and dosing solutions were made on the same day without any freezing of the core solution. In addition, the dosing solution was applied to the fabric for the malodor tracers on that same day.

TABLE 1
Malodor core solution composition

| | | | Weight |
|---|---|---|---|
| | | % | needed |
| Material | CAS | Composition | (g) |
| Undecanoic Acid | 112-37-8 | 66.7% | 2.25 |
| Decanoic Acid | 334-48-5 | 22.0% | 0.80 |
| Di-propylene glycol | 34590-94-8 | 15.3% | 0.55 |
| monomethyl ether | | | |

TABLE 2
Malodor dosing solution composition

| | | | Weight |
|---|---|---|---|
| | | % | needed |
| Material | CAS | Composition | (g) |
| Malodor Core Solution (Table 1) | — | 6.7% | 3.35 |
| Artificial Body Soil | — | 10% | 5.00 |
| Squalene | — | 10% | 5.00 |
| Di-propylene glycol | 34590-94-8 | 73.3% | 36.70 |
| monomethyl ether | | | |

### B. Preparation of Malodor Test Fabrics

The malodor tracer fabrics were polyester-cotton knit (PCW50/50), desized and cut into 0.8 in×1.0 in swatches, purchased from APD. For each tracer swatch, 250 μL of the malodor dosing solution was applied to the center.

For each test, ten malodor tracers and 25 g of white knitted cotton ballast were added to a tergotometer pot. The desized ballast fabrics were purchased from Warwick Equest Limited and were cut into 6 cm squares.

For each test, 2.37 grams of liquid detergent was added to tergotometer pots containing 1 L of wash solution plus malodor tracers and ballasts. The hardness of the test solution was adjusted to 7 US grains per gallon. The fabrics were agitated at 208 rpm for 12 minutes at 25° C. and spun dry. Fabrics were then rinsed in 15° C. water at 7 US grains per gallon at 200 rpm for 5 minutes and spun dry. After the rinse, fabrics were machine-dried and set on high for 20 minutes before being analysed.

The dried fabrics are placed in a mylar bag and sealed for 24 hours. Volatile malodor fatty acid species remaining on test fabrics were quantitatively determined by Gas Chromatography-Mass Spectroscopy.

### C. Analytical Detection of Malodor on Fabric Tracers

The malodor reduction using ABS/Squalene malodor sensors were quantitatively determined by Gas Chromatography Mass Spectroscopy using an Agilent gas chromatograph 7890B equipped with a mass selective detector (5977B), a MassHunter quantitation package and a Gerstel multi-purpose sampler equipped with a solid phase micro-extraction (SPME) probe. Calibration standards of 6-Methyl-5-hepten-2-one (CAS 110-93-0), Trans-2-heptenal (18829-55-5) and 3-methyl-2-Butenal (107-86-8) were prepared by dissolving a known weight of these materials in light mineral oil (CAS 8020-83-5) (each material available from Sigma Aldrich).

The washed malodor tracer fabrics were placed in 10 mL headspace crimp vials. Vials were equilibrated greater than 12 hours before analysis. The following settings were used in the autosampler: 80° C. incubation temperature, 45 min incubation time, VT15-10 sample tray type, 22 mm vial penetration, 2 min extraction time, 54 mm injection penetration and 300 s desorption time. The following settings were used for the Front Split/Splitless inlet helium: splitmode, 250° C. temperature, 9 psi pressure, 154.2 mL/min total flow, 3 mL/min septum purge flow, 125:1 split ratio and 15.44 min GC ran time. The following settings were used in the oven: 35° C. initial temperature, 16° C./min heating program, 250° C. temperature and 2 min hold time. Based on the partition coefficients (K at 80 C) of each component, the total nMol/L liter of 6-Methyl-5-hepten-2-one (K=3353), Trans-2-heptenal (K=3434), and 3-methyl-2-Butenal (K=1119) were calculated.

The values of these three measurements (in nanomoles/L) are added together to provide the Total ABS/Squalene Markers (nmol/L) for a given test leg.

### D. % Malodor Reduction Oxidation Products Calculations

The % Malodor Reduction Oxidation Products is provided as a percentage comparing the reduction of the amount of selected malodor markers as provided by the test composition compared to the (nil-chelant) reference composition. The value is determined as follows:

% Malodor Reduction=(Markers_{ref}−Markersₜₑₛₜ)×100/Markers_{ref}

Values for Markers_{ref} and Markersₜₑₛₜ are defined as follows:

Markers_{ref}=Total ABS/Squalene Markers (nanomoles/L) of the fabrics washed with the formulation without chelants (e.g., the reference or control formulation)

Markersₜₑₛₜ=Total ABS/Squalene Markers (nanomoles/L) of the fabrics washed with the formulation with the tested chelants

As the measured oxidation products are typically considered malodorous, it is believed that the greater the % reduction of oxidation products provided by composition, the less malodorous the treated fabrics are likely to be. Therefore, greater values of % Malodor.

Reduction Oxidation Products are typically preferred. The compositions and processes of the present disclosure may provide a % Malodor Reduction Oxidation Products value of at least about 10%, or at least about 20%, or at least about 30%, or at least about 40%, or at least about 50%, or at least about 60%, or at least about 70%, or at least about 80%.

### E. Quantification of Synergistic Effects

The synergistic effects could be formally defined as the following. For a measurable benefit, say, y, in this case, the % Malodor Reduction (% MR), for pure material 1 at weight fraction w₁, the measured benefit is y₁, and for pure material 2 at a weight fraction of w₂ the measured benefit is y₂. Without any synergy, one expects a mixture of material 1 at a weight fraction αw₁ and material 2 at weight fraction (1−α)w₂, yₘ=αy₁+(1−α)y₂, where α is a constant between 0 and 1.

The presence of synergy could be quantified by a parameter β, such that:

$${y}_{m} = α {y}_{1} + ( 1 - α ) {y}_{2} + 4 βα ( 1 - α ) ( {y}_{1 +} {y}_{2} )$$

This definition is convenient as the measurement at α=0.5 directly defines

$$β = ( 2 {y}_{m} ) / ( {y}_{1} + {y}_{2} ) - 1.$$

The fraction of deviation from the average defines the synergistic effect.

F. Malodor Reduction Synergistic Effects with Tetrasodium Glutamate Diacetate (GLDA) in HDL Formulations

| Raw Materials | Ref | A | B | C |
|---|---|---|---|---|
| Ethoxylated C10-16 linear alcohol¹ | 13.42 | 13.42 | 13.42 | 13.42 |
| Linear alkylbenzene sulfonate | 7.82 | 7.82 | 7.82 | 7.82 |
| Linear alkyl sulfate | 4.80 | 4.80 | 4.80 | 4.80 |
| Ethanol | 2.77 | 2.77 | 2.77 | 2.77 |
| Ethanolamine | 1.51 | 1.51 | 1.51 | 1.51 |
| Ethoxylated-Propoxylated | 1.00 | 1.00 | 1.00 | 1.00 |
| polyethyleneimine | | | | |
| C10-16-alkyldimethyl amine oxides | 0.96 | 0.96 | 0.96 | 0.96 |
| Linear alkyl ethoxylated sulfate | 0.87 | 0.87 | 0.87 | 0.87 |
| (MEA neutralized) | | | | |
| Propylene Glycol | 0.63 | 0.63 | 0.63 | 0.63 |
| Ethoxylated polyethyleneimine² | 0.62 | 0.62 | 0.62 | 0.62 |
| Sodium tetraborate | 0.59 | 0.59 | 0.59 | 0.59 |
| Hydrotrope | 0.58 | 0.58 | 0.58 | 0.58 |
| Ethoxylated C12-14 linear alcohols³ | 0.26 | 0.26 | 0.26 | 0.26 |
| Sodium Citrate | 0.25 | 0.25 | 0.25 | 0.25 |
| Sodium Formate | 0.24 | 0.24 | 0.24 | 0.24 |
| Tetrasodium Glutamate Diacetate | | 1 | 0.5 | |
| Ethyl Gallate | | | 0.5 | 1 |
| Water and minors (dyes, | balance | balance | balance | balance |
| preservatives, fragrances, etc.) | to 100% | to 100% | to 100% | to 100% |

¹Nonionic surfactant with an average degree of ethoxylation 7
²PE-20 commercially available from BASF
³Nonionic surfactant with an average degree of ethoxylation 9

### Notes:

The headspace measurement for this test is the following:

| | | | Malodor |
|---|---|---|---|
| | Markersₜₑₛₜ | Markers_{ref} | Reduction |
| Formulation | (nmol/L) | (nmol/L) | % |
| A | 203.3 ± 58.0 | 453.2 ± 26.1 | 55.1% |
| B | 133.6 ± 32.8 | 453.2 ± 26.1 | 70.5% |
| C | 339.4 ± 59.1 | 453.2 ± 26.1 | 25.1% |

The synergistic effect can be characterized by parameter β, defined in an earlier section. Here β(EG, GLDA)=0.758.

G. Malodor Reduction Synergistic Effects with Diethylenetriamine Pentaacetate (DTPA) in HDL Formulations

| Raw Materials | Ref | D | E | F |
|---|---|---|---|---|
| Ethoxylated C10-16 linear alcohol¹ | 13.42 | 13.42 | 13.42 | 13.42 |
| Linear alkylbenzene sulfonate | 7.82 | 7.82 | 7.82 | 7.82 |
| Linear alkyl sulfate | 4.80 | 4.80 | 4.80 | 4.80 |
| Ethanol | 2.77 | 2.77 | 2.77 | 2.77 |
| Ethanolamine | 1.51 | 1.51 | 1.51 | 1.51 |
| Ethoxylated-Propoxylated | 1.00 | 1.00 | 1.00 | 1.00 |
| polyethyleneimine | | | | |
| C10-16-alkyldimethyl amine | 0.96 | 0.96 | 0.96 | 0.96 |
| oxides | | | | |
| Linear alkyl ethoxylated sulfate | 0.87 | 0.87 | 0.87 | 0.87 |
| (MEA neutralized) | | | | |
| Propylene Glycol | 0.63 | 0.63 | 0.63 | 0.63 |
| Ethoxylated polyethyleneimine² | 0.62 | 0.62 | 0.62 | 0.62 |
| Sodium tetraborate | 0.59 | 0.59 | 0.59 | 0.59 |
| Hydrotrope | 0.58 | 0.58 | 0.58 | 0.58 |
| Ethoxylated C12-14 linear alcohol³ | 0.26 | 0.26 | 0.26 | 0.26 |
| Sodium Citrate | 0.25 | 0.25 | 0.25 | 0.25 |
| Sodium Formate | 0.24 | 0.24 | 0.24 | 0.24 |
| Diethylenetriamine Pentaacetate | | 1 | 0.5 | |
| Ethyl Gallate | | | 0.5 | 1 |
| Water and minors (dyes, | balance | balance | balance | balance |
| preservatives, fragrances, etc.) | to 100% | to 100% | to 100% | to 100% |

Notes:
¹Nonionic surfactant with an average degree of ethoxylation 7
²PE-20 commercially available from BASF
³Nonionic surfactant with an average degree of ethoxylation 9

The headspace measurement for this test is the following:

| | | | Malodor |
|---|---|---|---|
| | Markersₜₑₛₜ | Markers_{ref} | Reduction |
| Formulation | (nmol/L) | (nmol/L) | % |
| D | 174.7 ± 58.9 | 453.2 ± 26.1 | 61.5% |
| E | 58.0 ± 23.0 | 453.2 ± 26.1 | 87.2% |
| F | 339.4 ± 59.1 | 453.2 ± 26.1 | 25.1% |

The synergistic effect can be characterized by parameter β, defined in an earlier section. Here β(EG, DTPA)=1.014.

H. Malodor Reduction Synergistic Effects with Ethylenediamine-N,N′-Disuccinic Acid (EDDS) in HDL Formulations

| Raw Materials | Ref | G | H | I |
|---|---|---|---|---|
| Ethoxylated C10-16 linear alcohol¹ | 13.42 | 13.42 | 13.42 | 13.42 |
| Linear alkylbenzene sulfonate | 7.82 | 7.82 | 7.82 | 7.82 |
| Linear alkyl sulfate | 4.80 | 4.80 | 4.80 | 4.80 |
| Ethanol | 2.77 | 2.77 | 2.77 | 2.77 |
| Ethanolamine | 1.51 | 1.51 | 1.51 | 1.51 |
| Ethoxylated-Propoxylated | 1.00 | 1.00 | 1.00 | 1.00 |
| polyethyleneimine | | | | |
| C10-16-alkyldimethyl amine oxides | 0.96 | 0.96 | 0.96 | 0.96 |
| Linear alkyl ethoxylated sulfate | 0.87 | 0.87 | 0.87 | 0.87 |
| (MEA neutralized) | | | | |
| Propylene Glycol | 0.63 | 0.63 | 0.63 | 0.63 |
| Ethoxylated polyethyleneimine² | 0.62 | 0.62 | 0.62 | 0.62 |
| Sodium tetraborate | 0.59 | 0.59 | 0.59 | 0.59 |
| Hydrotrope | 0.58 | 0.58 | 0.58 | 0.58 |
| Ethoxylated C12-14 linear alcohol³ | 0.26 | 0.26 | 0.26 | 0.26 |
| Sodium Citrate | 0.25 | 0.25 | 0.25 | 0.25 |
| Sodium Formate | 0.24 | 0.24 | 0.24 | 0.24 |
| Ethylenediamine-N,N′-disuccinic | | 1 | 0.5 | |
| acid | | | | |
| Ethyl Gallate | | | 0.5 | 1 |
| Water and minors (dyes, | balance | balance | balance | balance |
| preservatives, fragrances, etc.) | to 100% | to 100% | to 100% | to 100% |

Notes:
¹Nonionic surfactant with an average degree of ethoxylation 7
²PE-20 commercially available from BASF
³Nonionic surfactant with an average degree of ethoxylation 9

The headspace measurement for this test is the following:

| | | | Malodor |
|---|---|---|---|
| | Markersₜₑₛₜ | Markers_{ref} | Reduction |
| Formulation | (nmol/L) | (nmol/L) | % |
| G | 240.7 ± 43.6 | 380.2 ± 19.8 | 36.7% |
| H | 142.0 ± 43.6 | 380.2 ± 19.8 | 62.7% |
| I | 309.5 ± 41.9 | 380.2 ± 19.8 | 18.6% |

The synergistic effect can be characterized by parameter β, defined in an earlier section. Here β(EG, EDDS)=1.268.

### Heavy Duty Liquid Example Compositions.

| | Comp. 1 | Comp. 2 | Comp. 3 | Comp. 4 | Comp. 5 | Comp. 6 | Comp 7 |
|---|---|---|---|---|---|---|---|
| Raw Material | % wt | % wt | % wt | % wt | % wt | % wt | % wt |
| Branched Alkyl | 0 | 0 | 15 | — | — | — | 0 |
| Sulfate¹² (BAS) | | | | | | | |
| Sodium Lauryl | — | 0 | 0 | 3.0 | 4.2 | 5.0 | 0 |
| Sulfate | | | | | | | |
| linear alkyl benzene | 4.3 | 7 | 4 | 3.0 | 12.9 | 8.3 | 12 |
| sulfonate | | | | | | | |
| AE3S Ethoxylated | 0 | 1.4 | 0 | — | — | — | 2.3 |
| alkyl sulfate with | | | | | | | |
| an average degree | | | | | | | |
| of ethoxylation of 3 | | | | | | | |
| C25AES | 1.36 | 0 | 2 | — | 2.5 | 0.9 | 0 |
| Ethoxylated alkyl | | | | | | | |
| sulfate with an | | | | | | | |
| average degree of | | | | | | | |
| ethoxylation of 2.5¹ | | | | | | | |
| amine oxide | 0.17 | 0.5 | 1.3 | 1.3 | 0.9 | 1.0 | 0 |
| Nonionic | 5.1 | 12.9 | 5.3 | 15 | 14.5 | 14 | 4.8 |
| Surfactant² | | | | | | | |
| citric acid | 0.24 | 0.12 | 3.4 | 1.2 | 0.8 | 0.26 | 2.7 |
| Sodium citrate | 0 | 0.64 | 0 | 0 | 0 | 0 | 0 |
| fatty acid | 0 | 2.4 | 2.9 | 1.6 | 0.5 | 0.15 | 2.1 |
| Mannanase | 0.0017 | 0.0017 | 0.0017 | 0.0017 | 0.0017 | 0.0017 | 0.0017 |
| Pectawash | 0.00343 | 0.0343 | 0.00342 | 0.00343 | 0.00343 | 0.00343 | 0.00343 |
| Amylase | 0.00766 | 0.0076 | 0.00766 | 0.00766 | 0.00766 | 0.00766 | 0.00766 |
| Protease | 0.007706 | 0.00706 | 0.07706 | 0.07706 | 0.07706 | 0.07706 | 0.07706 |
| calcium/sodium | 0 | 0 | 0.08 | 0.06 | 0 | 0.3 | 0 |
| formate | | | | | | | |
| Sodium/Calcium | 1.5 | 0.3 | 0.03 | 0 | 0.12 | 0 | 0.02 |
| Chloride | | | | | | | |
| Ethoxylated | 0.5 | 1.1 | 2.7 | 1.1 | 1.6 | 0.3 | 0 |
| polyethyleneimine⁴ | | | | | | | |
| Amphiphilic graft | 0 | 0 | 0 | 0 | 0 | 0 | 0.97 |
| copolymer | | | | | | | |
| Ethoxylated- | 0.8 | 0.8 | 1.1 | 1.5 | 1.9 | 1.0 | 0 |
| Propoxylated | | | | | | | |
| polyethyleneimine | | | | | | | |
| Zwitterionic | 0 | 0 | 0 | 0 | 0 | 0 | 0.55 |
| polyamine | | | | | | | |
| Graft polymer⁵ | 0.3 | 0.2 | 0.2 | 0 | 0 | 0.4 | 0.3 |
| DTPA chelant¹³ | 0.0 | 0.3 | 0.0 | 0.0 | 0.0 | 0.0 | 0.0 |
| GLDA chelant¹⁴ | 0.12 | 0 | 0.23 | 0.3 | 0.3 | 0.21 | 0 |
| DTPMP¹⁷ | 0 | 0 | 0 | 0 | 0 | 0 | 0.81 |
| Ethyl gallate | 0.2 | 0.2 | 0.2 | 0.2 | 0.3 | 0.4 | 0.2 |
| Fluorescent | 0 | 0.04 | 0.05 | 0 | 0.1 | 0.2 | 0.05 |
| Brightener⁶ | | | | | | | |
| Ethanol | 0.2 | 0 | 2.1 | 0.92 | 2.3 | 2.7 | 0.61 |
| propylene glycol | 1.3 | 0.33 | 6.7 | 1.4 | 0.87 | 0.7 | 1.1 |
| Glycerine | 0 | 0 | 0 | 0 | 0 | 0 | 0.1 |
| monoethanolamine | 1.1 | 0 | 0.6 | 0 | 3.35 | 1.93 | 0.21 |
| DETA¹⁵ | 0 | 0 | 0.05 | 0 | 0.06 | 0.05 | 0 |
| Antioxidant⁷ | 0 | 0 | 0.1 | 0 | 0.05 | 0.05 | 0 |
| Hygiene Agent⁸ | 0 | 0.04 | 0 | 0 | 0 | 0 | 0 |
| NaOH | 0.2 | 0 | 1.1 | 1.3 | 0.13 | 0 | 3.5 |
| NaCS | 0.09 | 0 | 0.15 | 0.53 | 0.3 | 0.6 | 1.87 |
| Hydrogenated | 0.08 | 0.12 | 0.12 | 0 | 0.08 | 0 | 0.26 |
| Castor Oil | | | | | | | |
| aesthetic dye | 0.02 | 0.006 | 0.017 | 0 | 0.01 | 0.01 | |
| Leuco dye¹⁶ | 0 | 0 | 0 | 0 | 0 | 0 | 0.03 |
| Perfume | 0.4 | 0.5 | 0.6 | 0 | 0.8 | 0.7 | 1.39 |
| Perfume | 0.5 | 0 | 0.1 | 0 | 0.3 | 0 | 0 |
| microcapsules | | | | | | | |
| silicone antifoam⁹ | 0 | 0.03 | 0 | 0 | 0.1 | 0.01 | 0.03 |
| Hueing dye¹⁰ | 0 | 0 | 0 | 0 | 0.3 | 0.25 | 0 |
| Sodium sulfate | 0 | 0.15 | 0 | 0 | 0 | 0 | 0.06 |
| preservative | 0.03 | 0.0003 | 0 | 0.01 | 0 | 0.01 | 0 |
| water & | balance | balance | balance | balance | balance | balance | balance |
| miscellaneous | | | | | | | |

¹C12-15EO2.5S AlkylethoxySulfate where the alkyl portion of AES includes from about 13.9 to 14.6 carbon atoms
²Surfonic L24-9 commercially available from Huntsman and/or Neodol 45-7 commercially available from Shell Chemicals
3. Nuclease enzyme is as claimed in co-pending European application 19219568.3
⁴PE-20 commercially available from BASF
⁵graft polymer is a polyalkylene oxide back-bone and side chains derived from N-vinylpyrrolidone (B), and side chains derived from vinyl ester (C) derived from a saturated monocarboxylic acid containing from 1 to 6 carbon atoms and/or a methyl or ethyl ester of acrylic or methacrylic acid as described in WO2020005476.
⁶Fluorescent Brightener is disodium 4,4′-bis{[4-anilino-6-morpholino-s-triazin-2-yl]-amino}-2,2′-stilbenedisulfonate or 2,2′-([1,1′-Biphenyl]-4,4′-diyldi-2,1-ethenediyl)bis-benzenesulfonic acid disodium salt.
⁷Antioxidant 1 is 3,5-bis(1,1-dimethylethyl)-4-hydroxybenzenepropanoic acid, methyl ester [6386-38-5]
⁸Hygiene Agent is agent is Tinosan HP 100 commercially available from BASF
⁹Silicone emulsion is Xiameter AFE-0110 or AF8017 supplied by Dow Corning
¹⁰Polymeric dyes such as described in WO2011/98355, US 2012/225803 Al, US 2012/090102 Al, U.S. Pat. No. 7,686,892 B2, and WO2010/142503, WO2011/045195, WO2010/148624, WO2010/102861, WO2011/098355, WO2012/163871, WO2012/26665, WO2012/119859 and WO2011/047987
11. Preservative is Benzisothiazolinone (BIT) and/or Methylisothiazolinone (MIT)
¹²BAS is a Branched 2-alkyl primary alkyl alcohol sulfate as described in U.S. Pat. No. 1,180,782
¹³DTPA is Diethylenetriaminepentaacetic acid
¹⁴GLDA is Glutamic acid diacetic acid
¹⁵DETA is Diethylenetriamine
¹⁶Leuco dyes as disclosed in U.S. Pat. No. 10,676,699 and U.S. Pat. No. 10,717,950
¹⁷DTPMP chelant is diethylene triamine penta(methyl phosphonic) acid

### Further Definitions and Cross-References

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A detergent composition comprising:
at least one component, according to Formula 7: 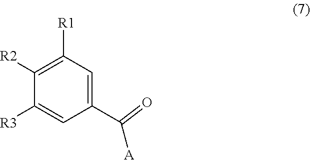
in which:
R₁₋₃ is an OH or an H and at least 2 adjacent R's are OH; and
A is an OR₄ or N(R₄)₂ or N(R₄)(H) or NH₂, where each R₄ is independently a linear or branched C₁₋₁₈ carbon chain, and
wherein the pH of the detergent composition is between about 6 and about 9.

2. The detergent composition according to claim 1, wherein the at least one component according to Formula 7 is selected from ethyl gallate, methyl gallate, propyl gallate, or a combination thereof.

3. The detergent composition according to claim 1, wherein the at least one component according to Formula 7 is ethyl gallate.

4. The detergent composition according to claim 3, further comprising a chelant selected from the group consisting of N,N-bis[2-[bis(carboxymethyl)amino]ethyl]glycine (CAS 67-43-6), N,N-bis(carboxymethyl)-L-Glutamic acid (CAS 58976-65-1), N,N′-1,2-ethanediylbis-L-Aspartic acid (CAS 20846-91-7), 2-hydroxy-4-(1-methylethyl)-2,4,6-Cycloheptatrien-1-one (CAS 499-44-5), P,P′,P″,P′″-[(phosphonomethyl)imino]bis[2,1-ethanediylnitrilobis(methylene)]]tetrakis-Phosphonic acid (CAS 15827-60-8), and other amino-carboxylate, phosphonic acid, and planar cyclic chelating materials, preferably N,N-bis[2-[bis(carboxymethyl)amino]ethyl]glycine (CAS 67-43-6), N,N-bis(carboxymethyl)-L-Glutamic acid (CAS 58976-65-1) and N,N′-1,2-ethanediylbis-L-Aspartic acid (CAS 20846-91-7).

5. The detergent composition according to claim 1, further comprising xyloglucanase.

6. The detergent composition according to claim 1, wherein the at least component according to Formula 7 has a pKa of less than about 9.5.

7. The detergent composition according to claim 1, wherein the at least one component according to Formula 7 is present in an amount of greater than 0 to about 10% by weight of the detergent composition.

8. The detergent composition according to claim 1, further comprising a surfactant comprising an anionic surfactant, a nonionic surfactant, a zwitterionic surfactant, amphoteric surfactant, cationic surfactant, or a combination thereof.

9. The detergent composition according to claim 8, wherein the surfactant is an anionic surfactant comprising at least one of alkyl ethoxylated sulfate, sodium lauryl sulfate, linear alkyl benzene sulfonic acid, branched 2-alkyl primary alkyl alcohol sulfate, alkyl sulphate, or a combination thereof.

10. The detergent composition according to claim 1, further comprising a graft copolymer, the graft copolymer comprising:
a. polyalkylene oxide which has a number average molecular weight of from about 1000 to about 20,000 Daltons and is based on ethylene oxide, propylene oxide, or butylene oxide;
b. N-vinylpyrrolidone; and
c. vinyl ester derived from a saturated monocarboxylic acid containing from 1 to 6 carbon atoms and/or a methyl or ethyl ester of acrylic or methacrylic acid;
wherein the weight ratio of (a) to (b) is from about 1:0.1 to about 1:2,
wherein the amount, by weight of the polymer, of (a) is greater than the amount of (c).

11. The detergent composition according to any claim 1, further comprising an antioxidant wherein the antioxidant is a hindered phenol.

12. The detergent composition according to claim 11, wherein the hindered phenol is 3,5-bis(1,1-dimethylethyl)-4-hydroxy-benzenepropanoic acid, methyl ester.

13. A single unit dose detergent composition comprising at least one component, according to Formula 7: 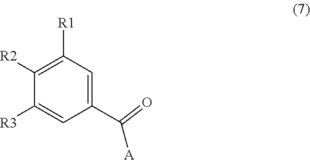
in which:
R₁₋₃ is an OH or an H and at least 2 adjacent R's are OH; and
A is an OR₄ or N(R₄)₂ or N(R₄)(H) or NH₂, where each R₄ is independently a linear or branched C1-18 carbon chain,
wherein the single unit dose is in the form of a water-soluble fibrous article.

14. A method for treating a stain on a fabric, comprising:
washing the fabric in a wash liquor comprising a detergent composition comprising at least one component, according to Formula 7: 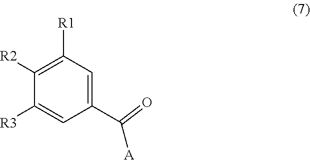
in which:
R₁₋₃ is an OH or an H and at least 2 adjacent R's are OH; and
A is a OR₄ or N(R₄)₂ or N(R₄)(H) or NH₂, where each R₄ is independently a linear or branched C1-18 carbon chain; and
wherein the pH of the detergent composition is between about 6 and about 9.

15. The method according to claim 14, wherein the at least one component according to Formula 7 is ethyl gallate, methyl gallate, propyl gallate, or a combination thereof.

16. The method according to claim 14, wherein the at least one component has a pKa of less than about 9.5.

17. The method according to claim 14, wherein the at least one component is present in an amount of from greater than 0 to about 10% by weight of the detergent composition.

18. The method according to claim 14, wherein the detergent composition is in the form or a liquid, gel, powder, bead, granule, fiber, or any combination thereof.

19. The method according to claim 14, wherein the stain comprises beverage, fruit, vegetable, or a combination thereof.

20. The method according to claim 14, wherein the detergent composition is in the form or a liquid, gel, powder, bead, granule, fiber, or any combination thereof.
